(19) United States
(12) Patent Application Publication
(10) Pub. No.: US 2023/0197216 A1
(43) Pub. Date: Jun. 22, 2023

(54) PERSONALIZED HEALTH ASSISTANT

(71) Applicant: SONY GROUP CORPORATION, Tokyo (JP)

(72) Inventors: SHYAM KISHORE, BANGALORE (IN); SWATHI VUDDALA, BANGALORE (IN); JYOTI SAHU, BANGALORE (JP)

(21) Appl. No.: 17/556,860

(22) Filed: Dec. 20, 2021

(51) Int. Cl. G16H 10/60; G16H 10/20; G06N 3/08

(52) U.S. Cl. CPC G16H 10/60; G16H 10/20; G06N 3/08

(57) ABSTRACT

A first electronic device and method for personalized health assistant is disclosed. The first electronic device receives user profile information associated with a user. The first electronic device receives a set of health templates based on the user profile information. The first electronic device selects a first health template from the received health templates. The first electronic device determines, by one or more sensors, a set of health parameters or a set of activities of the first user, based on the first health template. The first electronic device determines a set of health recommendations, based on application of a first artificial intelligence model on the first health template and on at the set of health parameters or the set of activities. The first electronic device controls a display device to render health information indicating the set of health recommendations and the set of health parameters or the set of activities.

### CROSS-REFERENCE TO RELATED APPLICATIONS/INCORPORATION BY REFERENCE

None

### FIELD

Various embodiments of the disclosure relate to personalized health assistant. More specifically, various embodiments of the disclosure relate to an electronic device and method for control of personalized health assistant.

### BACKGROUND

Advancements in technology have led to a proliferation of various electronic devices (such as, smart phones, smartwatches, activity trackers, and the like) with embedded sensors for fitness tracking. Also, a large number of software applications may be available for installation on such electronic devices for fitness tracking. Generally, a user may employ a dedicated software application associated with such electronic devices for a particular health goal. For example, a first software application may be employed to manage diet and workout plans, a second software application may be employed to track a sleep cycle, and a third software application may be employed to track water intake and other activities. In such a scenario, the user may have to spend some time to search for all such different software applications for different health goals and may have to individually configure each software application for fitness tracking.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method for control of personalized health assistant is provided substantially as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for control of a personalized health assistant, in accordance with an embodiment of the disclosure.

FIG. 2 is a block diagram that illustrates an exemplary first electronic device of FIG. 1, in accordance with an embodiment of the disclosure.

FIG. 3 is a block diagram that illustrates an exemplary server of FIG. 1, in accordance with an embodiment of the disclosure.

FIGS. 4A and 4B are diagrams that collectively illustrate an exemplary table for a set of health templates for users, in accordance with an embodiment of the disclosure.

FIGS. 5A and 5B are diagrams that collectively illustrate exemplary operations for control of a personalized health assistant, in accordance with an embodiment of the disclosure.

FIG. 6 is a diagram that illustrates an exemplary scenario for control of a personalized health assistant, in accordance with an embodiment of the disclosure.

FIG. 7 is a diagram that illustrates an exemplary scenario for display of a completion status of an activity, in accordance with an embodiment of the disclosure.

FIG. 8A is a diagram that illustrates an exemplary scenario for display of health information, in accordance with an embodiment of the disclosure.

FIG. 8B is a diagram that illustrates an exemplary scenario for graphical display of statistical information included in the health information, in accordance with an embodiment of the disclosure.

FIG. 9 is a flowchart that illustrates exemplary operations performed by the first electronic device (of FIG. 2) for control of a personalized health assistant, in accordance with an embodiment of the disclosure.

FIG. 10 is a flowchart that illustrates exemplary operations performed by the server (of FIG. 3) for control of a personalized health assistant, in accordance with an embodiment of the disclosure.

### DETAILED DESCRIPTION

The following described implementation may be found in a disclosed first electronic device and method for control of a personalized health assistant. Exemplary aspects of the disclosure provide a first electronic device (such as, but not limited to, a smartphone, a smartwatch, or an activity tracker), which may control a personalized health assistant. The first electronic device may be configured to receive user profile information associated with a first user of the first electronic device. The received user profile information may include, but is not limited to, a gender, an age, a height, a body weight, a body mass index (BMI), a location, food habits, medical data, or health goals of the first user. The first electronic device may be further configured to receive a set of health templates, based on the received user profile information associated with the first user. Thereafter, the first electronic device may be configured to select a first health template from the received set of health templates. By way of example, and not limitation, the set of health templates may be generated based on a user input received from a set of health practitioners or an application of an artificial intelligent (AI) model on the received user profile information. Each of the set of health templates may indicate a set of activities (for example, exercises, sleeping pattern, breaks from work, etc) and a set of health-related recommendations (for example diet plans) for the first user of the first electronic device. As the set of health templates may be generated based on the AI model or the user input from the health practitioners, the set of health templates may cover holistic health goals (such as related to physical health, social health, and/or mental health) of the first user. Thus, the set of health templates may correspond to customized health templates for the first user, such that the first health template may be selected from the set of health templates to further personalize the holistic health tracking of the first user with minimal effort.

Based on the selected first health template, the first electronic device may be configured to determine, by one or more sensors associated with the first electronic device, at least one of a set of health parameters of the first user or a set of activities of the first user. For example, the set of health parameters may include, but is not limited to, a body temperature, a heart rate, a pulse rate, a blood oxygen level, a blood pressure, a blood sugar level, a stress level, a sleep duration, or a depth of sleep. Examples of the set of activities may include, but are not limited to, a water intake, a food intake activity, a sleep, a step count, a meditation activity, a yoga activity, a physical exercise, a breathing exercise, a stretching exercise, a sedentary task, a walk, a run, a jog, a cycling activity, a swimming activity, a work-out activity, or a listening to music activity.

The first electronic device may be further configured to determine a set of health recommendations associated with the first user, based on an application of a first artificial intelligence (AI) model on the selected first health template and on at least one of the determined set of health parameters of the first user or the determined set of activities of the first user. By way of example, and not limitation, the determined set of health recommendations may include a recommendation for a first activity for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the first user.

The first electronic device may be further configured to set-up periodic auto-reminders, associated with the set of activities for the first user, based on the selected first health template. The first electronic device may automatically generate a notification associated with a first activity of the set of activities based on the set periodic auto-reminders and may control a display screen to display the notification. Also, the first electronic device may determine a completion status of the first activity of the first user based on at least one of the determined set of activities of the first user or a user input received from the first user. The first electronic device may further control the display device to display the determined completion status of the first activity.

The disclosed first electronic device may further control the display device associated with the first electronic device to render health information (e.g., a health dashboard). Such health information may be indicative of the set of health recommendations and at least one of the set of health parameters or the set of activities associated with the first user. By way of example, and not limitation, the health information may indicate statistical information associated with a health of the first user. The statistical information associated with the health may indicate, but not limited to, an amount of activities performed, an amount of expended calories, information about an endurance, an amount of food intake, an amount of calorie intake, nutrients of consumed food, ingredients of consumed food, an amount of water intake, or a weight change of the first user.

The disclosed first electronic device may provide the set of health templates (for example, top 5 health templates) for the first user based on the user profile information of the first user. The set of health templates may be curated by health practitioners or produced by an AI engine. Thus, the set of health templates may be tailor-made for the first user and may cover holistic health goals for the first user. The first health template may be selected from the set of health templates by the first user. As the set of health templates may be already customized for the first user, the selection of the first health template may be a mere personalization (based on preferences of the first user), which may be made easily, with minimal effort by the first user. The first electronic device may further set periodic auto-reminders for activities of the first user based on the selected first health template and may also determine the completion status of the activities. Thus, the activities of the first user may be seamlessly tracked without much configuration effort on part of the first user. The first electronic device may also determine AI-based recommendations for the first user based on the selected first health template, tracked activities, and tracked health parameters. Such recommendations may be used for further update of the selected first health template and may also help to improve health and fitness of the first user over a period of time. Further, the first electronic device may control rendering of the health information (such as, a health dashboard) including statistical information associated with health of the first user. The statistical information may include the recommendations, the tracked activities of the first user, and/or the tracked health parameters of the first user. The health information may serve as a consolidated cross-functional dashboard including insights that may help to track the health of the first user and also to ascertain a progress of the first user with respect to the desired health goals of the first user, as the selected health template may be personalized for the first user. Also, the first user may be spared from the effort associated with search of several different software applications and health-related devices for health tracking, as the disclosed first electronic device may provide a holistic health tracking experience to the first user.

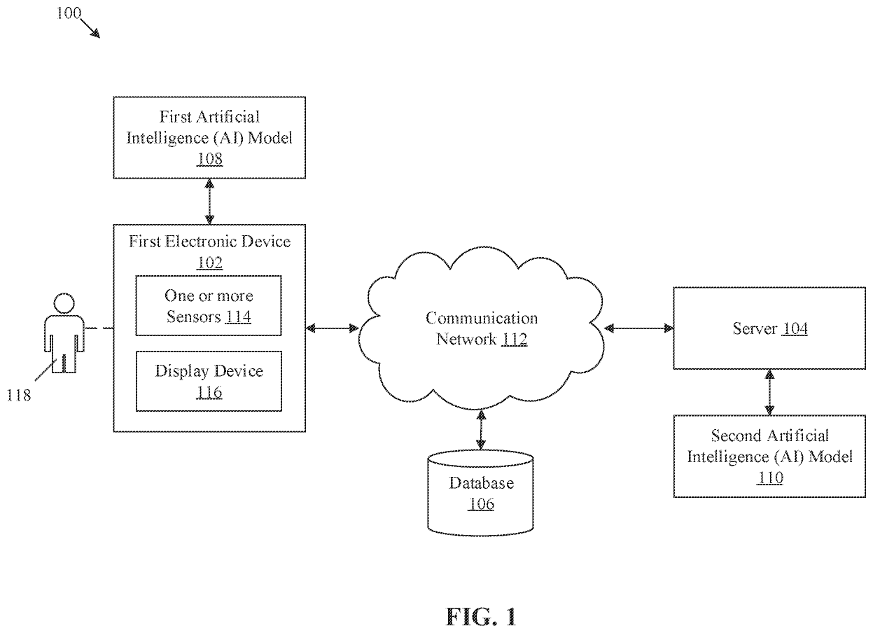

FIG. 1 is a block diagram that illustrates an exemplary network environment for control of a personalized health assistant, in accordance with an embodiment of the disclosure. With reference to FIG. 1, there is shown a network environment 100. The network environment 100 may include a first electronic device 102, a server 104, a database 106, a first artificial intelligence (AI) model 108, and a second AI model 110. The first electronic device 102 may include one or more sensors 114, and a display device 116. The first electronic device 102, the server 104, and the database 106 may be communicatively coupled with each other, via a communication network 112. In the network environment 100, there is further shown a first user 118, who may be associated with the first electronic device 102.

The first electronic device 102 may include suitable logic, circuitry, interfaces, and/or code that may be configured to receive user profile information associated with the first user 118 and transmit the received user profile information to the server 104. The first electronic device 102 may receive a set of health templates from the server 104, based on the transmitted user profile information, and may select a first health template from the received set of health templates. The first electronic device 102 may be further configured to control the display device 116 to render health information associated with the first user 118 on the display device 116. The health information may indicate a set of health recommendations and at least one of a set of health parameters or a set of activities associated with the first user 118. Examples of the first electronic device 102 may include, but are not limited to, a smartphone, a wearable computing device (such as, a smartwatch or an activity tracker), a mobile phone, a tablet, a laptop, a gaming device, a handheld device (such as, a tablet computing device), a wearable haptic device, a head-mounted display (such as an eXtended Reality (XR) display, or a helmet with a Head-up Display (HUD) or an integrated display panel).

The one or more sensors 114 may be associated with the first electronic device 102 and may include suitable logic, circuitry, code, and/or interfaces that may be configured to determine at least one of the set of health parameters of the first user 118 or the set of activities of the first user 118, based on instructions received from the first electronic device 102. Examples of the one or more sensors 114 may include, but are not limited to, an accelerometer, an altimeter, a gyroscope, a step-count tracker, a heart rate tracker, a pulse rate monitor, a blood oxygen concentration monitor, a bioimpedance sensor, a location sensor, an activity tracker, an ultra-violet (UV) sensor, an electrodermal activity sensor, a skin temperature sensor, an electrocardiography sensor, a gesture sensor, or a magnetometer. In an embodiment, the one or more sensors 114 may be external to the first electronic device 102 and may be communicatively coupled to the first electronic device 102. In another embodiment, the one or more sensors 114 may be embedded or inbuilt within the first electronic device 102, and thus may be integrated with the first electronic device 102.

The display device 116 may include suitable logic, circuitry, and/or interfaces that may be configured to render the health information generated by the first electronic device 102. For example, the display device 116 may be utilized to display the set of health recommendations and at least one of the determined set of health parameters or the determined set of activities associated with the first user 118. The display device 116 may be further utilized to display a completion status of the set of activities of the first user 118 based on at least one of the determined set of activities of the first user 118 or a user input received from the first user 118. In an embodiment, the display device 116 may be externally coupled with the first electronic device 102 via an I/O interface or a network interface. In another embodiment, the display device 116 may be integrated into the first electronic device 102.

In at least one embodiment, the display device 116 may be a touch screen which may enable the first user 118 to provide a user input via the display device 116. The touch screen may be at least one of a resistive touch screen, a capacitive touch screen, or a thermal touch screen. The display device 116 may be realized through several known technologies such as, but not limited to, a Liquid Crystal Display (LCD) display, a Light Emitting Diode (LED) display, a plasma display, or an Organic LED (OLED) display, or other display technologies. In accordance with an embodiment, the display device 116 may refer to a display screen of a head mounted device (HMD), a smart-glass device, a see-through display, a projection-based display, an electro-chromic display, or a transparent display.

The server 104 may include suitable logic, circuitry, and interfaces, and/or code that may be configured to store a plurality of health templates and a trained artificial intelligence (AI) model (such as, the second AI model 110). The server 104 may be configured to determine the set of health templates from the stored plurality of health templates based on an application of the second AI model 110 on the user profile information received from the first electronic device 102. The server 104 may further transmit the determined set of health templates to the first electronic device 102. Each of the determined set of health templates may indicate the set of activities and the set of health recommendations for the first user 118 of the first electronic device 102. In certain embodiments, the server 104 may receive the selected first health template and periodically determined values of the set of health parameters and/or the set of activities of the first user 118 from the first electronic device 102. The server 104 may apply the second AI model 110 on the received first health template and at least one of the received set of health parameters and/or the set of activities of the first user 118 to determine health recommendations for the first user 118. The server 104 may further transmit the determined health recommendations to the first electronic device 102, for display on the display device 116.

In an exemplary embodiment, the server 104 may be implemented as a cloud server and may execute operations through web applications, cloud applications, HTTP requests, repository operations, file transfer, and the like. Other example implementations of the server 104 may include, but are not limited to, a database server, a file server, a content server, a web server, an application server, a mainframe server, or a cloud computing server. In at least one embodiment, the server 104 may be implemented as a plurality of distributed cloud-based resources by use of several technologies that are well known to those ordinarily skilled in the art. A person with ordinary skill in the art will understand that the scope of the disclosure may not be limited to the implementation of the server 104 and the first electronic device 102 as two separate entities. In certain embodiments, the functionalities of the server 104 may be incorporated in its entirety or at least partially in the first electronic device 102, without a departure from the scope of the disclosure.

The first AI model 108 may be trained on a health recommendation determination task and/or a health condition determination task. The first AI model 108 may be configured to determine the set of health recommendations for the first user 118 based on inputs, such as, the selected first health template and at least one of the determined set of health parameters of the first user 118 or the determined set of activities of the first user 118. Also, the first AI model 108 may be configured to determine a health condition of the first user 118 based on at least one of the determined set of health parameters of the first user 118 or the determined set of activities of the first user 118.

The second AI model 110 may be trained on a health template determination task and/or a health recommendation determination task. The second AI model 110 may be configured to determine the set of health templates from the stored plurality of health templates based on inputs, such as, the user profile information associated with the first user 118. Also, the second AI model 110 may be configured to determine health recommendations for the first user 118 based on the selected first health template and at least one of the determined set of health parameters of the first user 118 or the determined set of activities of the first user 118.

In an embodiment, each of the first AI model 108 the second AI model 110 may be implemented as a neural network model, such as, a deep learning model. The neural network model may be defined by its hyper-parameters and topology/architecture. For example, the neural network model may be a deep neural network-based model that may have a number of nodes (or neurons), activation function(s), number of weights, a cost function, a regularization function, an input size, a learning rate, number of layers, and the like. Such a model may be referred to as a computational network or a system of nodes (for example, artificial neurons). For a neural network implementation, the nodes of the neural network model may be arranged in layers, as defined in a neural network topology. The layers may include an input layer, one or more hidden layers, and an output layer. Each layer may include one or more nodes (or artificial neurons, represented by circles, for example). Outputs of all nodes in the input layer may be coupled to at least one node of hidden layer(s). Similarly, inputs of each hidden layer may be coupled to outputs of at least one node in other layers of the model. Outputs of each hidden layer may be coupled to inputs of at least one node in other layers of the neural network model. Node(s) in the final layer may receive inputs from at least one hidden layer to output a result. The number of layers and the number of nodes in each layer may be determined from the hyper-parameters, which may be set before, while, or after training the neural network model on a training dataset.

Each node of the neural network model may correspond to a mathematical function (e.g., a sigmoid function or a rectified linear unit) with a set of parameters, tunable during training of the model. The set of parameters may include, for example, a weight parameter, a regularization parameter, and the like. Each node may use the mathematical function to compute an output based on one or more inputs from nodes in other layer(s) (e.g., previous layer(s)) of the neural network model. All or some of the nodes of the neural network model may correspond to same or a different mathematical function.

In training of the neural network model, one or more parameters of each node may be updated based on whether an output of the final layer for a given input (from the training dataset) matches a correct result based on a loss function for the neural network model. The above process may be repeated for the same or a different input till a minima of loss function is achieved, and a training error is minimized. Several methods for training are known in the art, for example, gradient descent, stochastic gradient descent, batch gradient descent, gradient boost, meta-heuristics, and the like.

In an embodiment, each of the first AI model 108 and the second AI model 110 may include electronic data, which may be implemented as, for example, a software component of an application executable on a computing device (such as, the first electronic device 102 or the server 104, respectively). Each of the first AI model 108 and the second AI model 110 may include code and routines that may be configured to enable a computing device, such as, the first electronic device 102 or the server 104, respectively, to perform one or more operations for determination of the set of health recommendations. Additionally, or alternatively, each of the first AI model 108 and the second AI model 110 may be implemented using hardware including, but not limited to, a processor, a microprocessor (e.g., to perform or control performance of one or more operations), a field-programmable gate array (FPGA), a co-processor (such as an AI-accelerator), or an application-specific integrated circuit (ASIC). In some embodiments, each of the trained first AI model 108 and the trained second AI model 110 may be implemented using a combination of both hardware and software.

In certain embodiments, each of the first AI model 108 and the second AI model 110 may be based on a hybrid architecture of multiple Deep Neural Networks (DNNs). Examples of each of the first AI model 108 and the second AI model 110 may include, but are not limited to, a neural network model or a model based on one or more of regression method(s), instance-based method(s), regularization method(s), decision tree method(s), Bayesian method(s), clustering method(s), association rule learning, and dimensionality reduction method(s). Examples of the neural network model may include, but are not limited to, an artificial neural network (ANN), a deep neural network (DNN), a convolutional neural network (CNN), a recurrent neural network (RNN), a CNN-recurrent neural network (CNN-RNN), R-CNN, Fast R-CNN, Faster R-CNN, a Residual Neural Network (Res-Net), a Feature Pyramid Network (FPN), and/or a combination thereof.

The database 106 may be configured to store the plurality of health templates. In an embodiment, the database 106 may be configured to store the user profile information associated with the first user 118. The user profile information associated with the first user 118 may include, but is not limited to at least one of a gender, an age, a height, a body weight, a body mass index (BMI), a location, food habits, medical data, or health goals of the first user 118. The database 106 may store user profile information associated with each of a plurality of users associated with a corresponding electronic device (not shown in FIG. 1) of the network environment 100. The database 106 may be derived from data off a relational or non-relational database, or a set of comma-separated values (csv) files in conventional or big-data storage. The database 106 may be stored on a server, such as the server 104 or may be cached and stored on the first electronic device 102. The device storing the database 106 may be configured to receive a query for plurality of health templates and/or the user profile information of the first user 118 from the server 104. In response, the device of the database 106 may be configured to retrieve and provide the queried plurality of health templates and/or the user profile information of the first user 118, based on the received query.

In some embodiments, the database 106 may be hosted on a plurality of servers stored at same or different locations. The operations of the database 106 may be executed using hardware including a processor, a microprocessor (e.g., to perform or control performance of one or more operations), a field-programmable gate array (FPGA), or an application-specific integrated circuit (ASIC). In some other instances, the database 106 may be implemented using software.

The communication network 112 may include a communication medium, through which the first electronic device 102, the server 104, and the database 106 may communicate with each other. Examples of the communication network 112 may include, but are not limited to, the Internet, a cloud network, a Wireless Local Area Network (WLAN), a Wireless Fidelity (Wi-Fi) network, a Personal Area Network (PAN), a Local Area Network (LAN), a telephone line (POTS), and/or a Metropolitan Area Network (MAN), a mobile wireless network, such as a Long-Term Evolution (LTE) network (for example, 4th Generation or 5th Generation (5G) mobile network (i.e., 5G New Radio)). Various devices in the network environment 100 may be configured to connect to the communication network 112, in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), ZigBee, EDGE, IEEE 802.11, light fidelity (Li-Fi), 802.16, IEEE 802.11s, IEEE 802.11g, multi-hop communication, wireless access point (AP), device to device communication, cellular communication protocols, or Bluetooth (BT) communication protocols, or a combination thereof.

In operation, the first electronic device 102 may receive a user input, for example, to turn-on the first electronic device 102 or to activate a hardware or software application associated with a personalized health assistant in the first electronic device 102. In such a scenario, the first electronic device 102 may be configured to perform a set of operations to control the hardware or software application associated with the personalized health assistant. A description of such operations is described herein.

In an example, the first electronic device 102 may correspond to a smartphone communicably coupled to a wearable electronic device (such as, a smartwatch or an activity tracker) worn by the first user 118. In such a case, the first electronic device 102 may control the display device 116 to display a user interface. The user interface may be an interface of a smartphone application that may be configured to display options to view various information, such as the set of health templates, the set of health parameters, the set of activities, and the set of health recommendations. Examples of the user interface may include, but is not limited to, a graphical user interface (GUI) of a software application that may be installed on the first electronic device 102 or may be accessible via a web client of the first electronic device 102. The software application may send or receive data to or from various sources, such as, the one or more sensors 114, the wearable electronic device, a server that stores data of the first user 118, and/or third-party data aggregators.

In another example, the first electronic device 102 may correspond to a wearable electronic device (such as, a smartwatch, or an activity tracker). The wearable electronic device may include suitable logic, circuitry, and interfaces that may be configured to receive a touch input from the first user 118. In an embodiment, the wearable electronic device may be in contact with at least one first anatomical portion of a body (e.g., a wrist or ankle) of the first user 118. In another embodiment, the wearable electronic device may be a wrapped, wound, or strapped around the first anatomical portion of the body. The wearable electronic device may include the one or more sensors 114.

At a time instance, the first electronic device 102 may be configured to receive user profile information associated with the first user 118 of the first electronic device 102. Examples of the received user profile information may include, but are not limited to, a gender, an age, a height, a body weight, a body mass index (BMI), a location, food habits, medical data, or health goals of the first user 118. In an embodiment, the first electronic device 102 may be configured to transmit the received user profile information to the server 104.

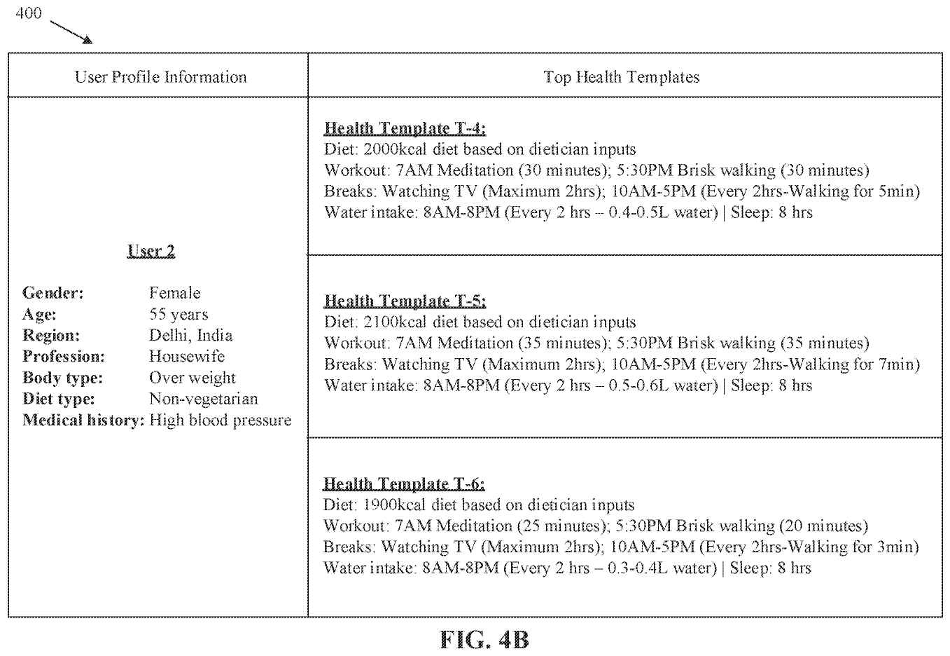

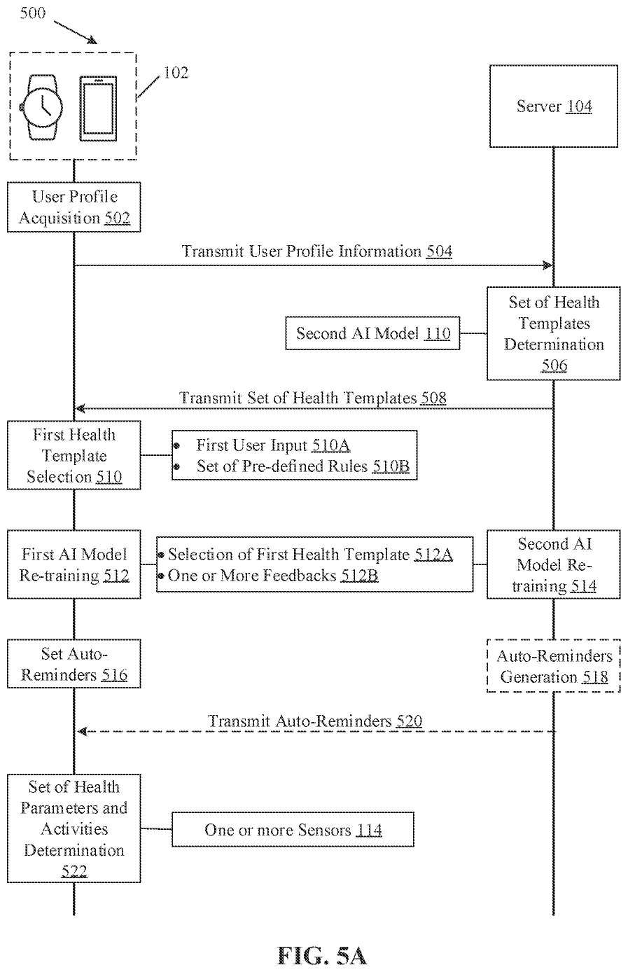

The first electronic device 102 may be configured to receive a set of health templates based on the received user profile information associated with the first user 118. In an embodiment, the set of health templates may be received from the server 104. In an embodiment, each of the determined set of health templates may indicate a set of activities and a set of health recommendations for the first user 118 of the first electronic device 102. Thereafter, the first electronic device 102 may be configured to select a first health template from the received set of health templates. Details related to the set of health templates are provided, for example, in FIGS. 4A and 4B. The first electronic device 102 may be configured to determine, by the one or more sensors 114 associated with the first electronic device 102, at least one of a set of health parameters of the first user 118 or a set of activities of the first user 118, based on the selected first health template. For example, the set of health parameters may include, but are not limited to, a body temperature, a heart rate, a pulse rate, a blood oxygen level, a blood pressure, a blood sugar level, a stress level, a sleep duration, or a depth of sleep. Examples for the set of activities may include, but not limited to a water intake activity, a food intake activity, a sleep activity, a step count, a meditation activity, a yoga activity, a physical exercise, a breathing exercise, a stretching exercise, a sedentary task, a walk, a run, a jog, a cycling activity, a swimming activity, a work-out activity, or a listening to music activity. Details related to the determination of the set of health parameters, or the set of activities are provided, for example, in FIGS. 5A and 5B.

The first electronic device 102 may be configured to determine a set of health recommendations associated with the first user 118, based on an application of the first artificial intelligence (AI) model 108 on the selected first health template and on at least one of the determined set of health parameters of the first user 118 or the determined set of activities of the first user 118. For example, the determined set of health recommendations may include, but is not limited to, a recommendation for a first activity for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the first user 118. The determination of the set of health recommendations is described further, for example, in FIGS. 5A and 5B.

The first electronic device 102 may be configured to control the display device 116 associated with the first electronic device 102 to render health information (e.g., a health dashboard). The health information may indicate the determined set of health recommendations and at least one of the determined set of health parameters or the determined set of activities associated with the first user 118. The control of the display device 116 to render the health information is described further, for example, in FIGS. 5A and 5B. By way of example, and not limitation, the health information may indicate statistical information associated with a health of the first user 118. Herein, the statistical information associated with the health may comprise at least one of, but not limited to, an amount of activities performed, an amount of expended calories, information about an endurance, an amount of food intake, an amount of calorie intake, nutrients of consumed food, ingredients of consumed food, an amount of water intake, or a weight change.

The selected first health template may cover holistic health goals (such as related to physical health, social health, and/or mental health) of the first user 118, based on the user profile information of the first user 118. As the set of health templates may be already customized for the first user 118 based on the user profile information of the first user 118, the selection of the first health template may be a mere personalization (based on a preference of the first user 118), which may be made with minimal effort by the first user 118. The selected first health template may help the first user 118 to achieve desired health goals as the selected health template may be personalized for the first user 118. The first electronic device 102 may also determine AI-based recommendations for the first user 118 based on the selected first health template, the determined set of activities, and determined set of health parameters. Such recommendations may be used for further update of the selected first health template and may also help to improve health and fitness of the first user 118 over a period of time. Further, the first electronic device 102 may control rendering of health information (such as, a health dashboard) including statistical information associated with the health of the first user 118. The statistical information may include the recommendations, the determined set of activities, and/or the determined health parameters of the first user 118. The health information may serve as a consolidated cross-functional dashboard including insights that may help to track the health of the first user 118 and also to ascertain a progress of the first user 118 with respect to the desired health goals of the first user 118. Also, the first user 118 may be spared from the effort associated with search of several different software applications and health-related devices for health tracking, as the first electronic device 102 may provide a holistic health tracking experience to the first user 118.

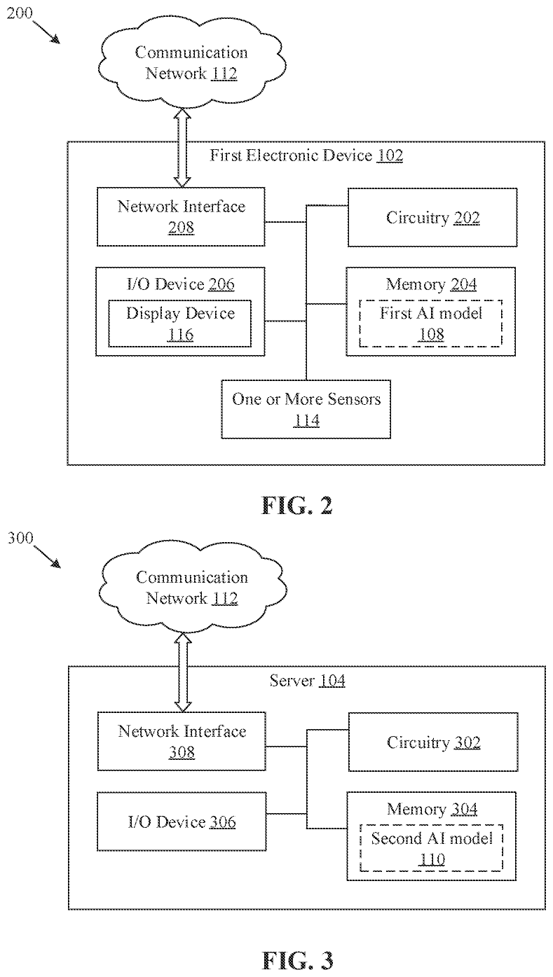

FIG. 2 is a block diagram that illustrates an exemplary first electronic device of FIG. 1, in accordance with an embodiment of the disclosure. FIG. 2 is explained in conjunction with elements from FIG. 1. With reference to FIG. 2, there is shown the first electronic device 102. The first electronic device 102 may include circuitry 202, a memory 204, an input/output (I/O) device 206, a network interface 208, and the one or more sensors 114. The I/O device 206 may include the display device 116. The memory 204 may include the first artificial intelligence (AI) model 108. The network interface 208 may connect the first electronic device 102 with the server 104 and the database 106, via the communication network 112.

The circuitry 202 may include suitable logic, circuitry, and/or interfaces that may be configured to execute program instructions associated with different operations to be executed by the first electronic device 102. The operations may include, but are not limited to, reception of the user profile information, reception of the set of health templates, selection of the first health template, determination of at least one of the set of health parameters, or the set of activities of the first user 118, determination of the set of health recommendations, and control of the display device 116. The circuitry 202 may include one or more processing units, which may be implemented as a separate processor. In an embodiment, the one or more processing units may be implemented as an integrated processor or a cluster of processors that perform the functions of the one or more specialized processing units, collectively. The circuitry 202 may be implemented based on a number of processor technologies known in the art. Examples of implementations of the circuitry 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the circuitry 202. The memory 204 may be configured to store the first AI model 108, and the set of health templates. The memory 204 may be further configured to store the user profile information associated with the first user 118. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. The I/O device 206 may include various input and output devices, which may be configured to communicate with the circuitry 202. In an example, the first electronic device 102 may receive (via the I/O device 206) the user input indicative of the user profile information associated with the first user 118. In an example, the first electronic device 102 may display (via the display device 116 associated with the I/O device 206) the health information. Examples of the I/O device 206 may include, but are not limited to, a touch screen, a keyboard, a mouse, a joystick, a display device (for example, the display device 116), a microphone, or a speaker.

The network interface 208 may include suitable logic, circuitry, interfaces, and/or code that may be configured to facilitate communication between the first electronic device 102, the server 104, and the database 106, via the communication network 112. The network interface 208 may be implemented by use of various known technologies to support wired or wireless communication of the first electronic device 102 with the communication network 112. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a protocol for email, instant messaging, and a Short Message Service (SMS). Various operations of the circuitry 202 for personalized health assistant control are described further, for example, in FIGS. 5A, 5B, 6, 7, 8, and 9.

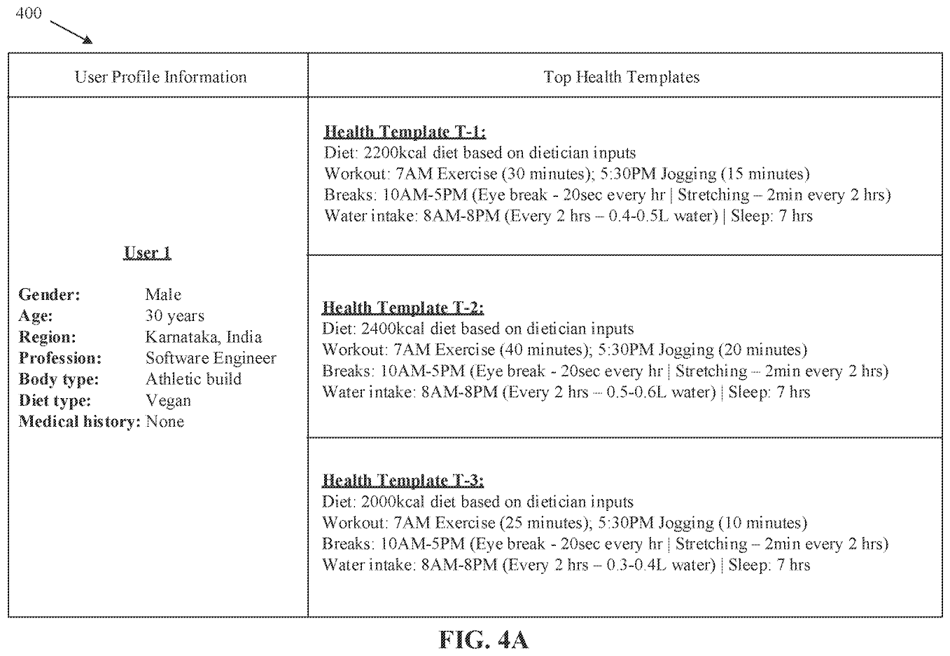

FIG. 3 is a block diagram that illustrates an exemplary server of FIG. 1, in accordance with an embodiment of the disclosure. FIG. 3 is explained in conjunction with elements from FIGS. 1 and 2. With reference to FIG. 3, there is shown a block diagram 300 of the server 104. The server 104 may include circuitry 302, a memory 304, an Input/Output (I/O) device 306, and a network interface 308. The memory 304 may include the second AI model 110. The network interface 308 may connect the server 104 with the first electronic device 102 and the database 106, via the communication network 112.

The circuitry 302 may include suitable logic, circuitry, interfaces, and/or code that may be configured to execute program instructions associated with different operations to be executed by the server 104. The operations may include, but are not limited to, reception of the user profile information associated with the first user 118, determination of the set of health templates, transmission of the determined set of health templates, reception of the set of activities or the set of health parameters of the first user 118, determination of the set of health recommendations, and transmission of the determined set of health recommendations. The circuitry 302 may include any suitable special-purpose or general-purpose computer, computing entity, or processing device including various computer hardware or software modules and may be configured to execute instructions stored on any applicable computer-readable storage media. For example, the circuitry 302 may include a microprocessor, a microcontroller, a DSP, an ASIC, a FPGA, or any other digital or analog circuitry configured to interpret and/or to execute program instructions and/or to process data. The functions of the circuitry 302 may be same as the functions of the circuitry 202 described, for example, in FIG. 2. Therefore, further description of the circuitry 302 is omitted from the disclosure for the sake of brevity.

The memory 304 may include suitable logic, circuitry, interfaces, and/or code that may be configured to store program instructions to be executed by the circuitry 302. In certain embodiments, the memory 304 may be configured to store operating systems and associated application-specific information. In at least one embodiment, the memory 304 may be configured to store the second AI model 110. The functions of the memory 304 may be same as the functions of the memory 204 described, for example, in FIG. 2. Therefore, further description of the memory 304 is omitted from the disclosure for the sake of brevity.

The I/O device 306 may include suitable logic, circuitry, interfaces, and/or code that may be configured to receive an input and provide an output based on the received input. The I/O device 306 may include various input and output devices, which may be configured to communicate with the circuitry 302. The functions of the I/O device 306 may be same as the functions of the I/O device 206 described, for example, in FIG. 2. Therefore, further description of the I/O device 306 is omitted from the disclosure for the sake of brevity.

The network interface 308 may include suitable logic, circuitry, interfaces, and/or code that may enable communication between the server 104, first electronic device 102 and the database 106, via the communication network 112. The network interface 308 may implement known technologies to support wired and/or wireless communication. The network interface 308 may include, but is not limited to, an antenna, a frequency modulation (FM) transceiver, a radio frequency (RF) transceiver, one or more amplifiers, a tuner, one or more oscillators, a digital signal processor, a coder-decoder (CODEC) chipset, a subscriber identity module (SIM) card, and/or a local buffer. The functions of the network interface 308 may be same as the functions of the network interface 208 described, for example, in FIG. 2. Therefore, further description of the network interface 308 is omitted from the disclosure for the sake of brevity.

It should be noted that some or all of the functions and/or operations performed by the circuitry 202 (as described in FIG. 2) may be performed by the circuitry 302, without a deviation from the scope of the disclosure.

FIGS. 4A and 4B are diagrams that collectively illustrate an exemplary table for a set of health templates for users, in accordance with an embodiment of the disclosure. FIGS. 4A and 4B are explained in conjunction with elements from FIGS. 1, 2, and 3. With reference to FIGS. 4A and 4B, there is shown a table 400. The table 400 may include columns such as user profile information of users, and top health templates for respective users. The table 400 may include multiple rows, where each row may correspond to a set of health templates related to a respective user (such as, “User 1” and “User 2”).

By way of example, and not limitation, the user profile information of the first user 118 may include, but is not limited to, a gender, an age, a height, a body weight, a body mass index (BMI), a location, food habits, medical data, or health goals of the first user 118. The location may correspond to a geolocation of the first user 118. For example, the first electronic device 102 may be configured to receive a user input indicative of information associated with the location of the first user 118. The user input may specify a country, a state, a city, a province, a postal code, or a zip code where the first user 118 may be residing or located. In some embodiments, the first electronic device 102 may include a location sensor (not shown) as the one or more sensors 114. The location sensor may include logic, circuitry, code and/or interfaces that may be configured to acquire the information associated with the location of the first user 118. The location sensor of the first electronic device 102 may be configured to transmit the acquired information to the circuitry 202. Examples of the location sensor may include, but are not limited to, a Global Navigation Satellite System (GNSS)-based sensor and a mobile positioning system (such as a system that uses LTE positioning protocol).

The food habits may correspond to eating habits of the first user 118. For example, the first electronic device 102 may be configured to receive a user input indicative of information associated with the food habits of the first user 118. The user input may specify a type of diet generally followed by the first user 118, such as, but not limited to, a vegetarian diet, a vegan diet, a non-vegetarian diet, a ketogenic diet, or a gluten-free diet. The medical data may correspond to healthcare data of the first user 118. For example, the first electronic device 102 may be configured to receive a user input indicative of information associated with the medical data of the first user 118. The user input may specify a health-related issues of the first user 118, such as, but not limited to, a heart disease, diabetes, obesity, anxiety, depression, or allergies. The health goals may correspond to lifestyle goals of the first user 118 to maintain and/or improve health. For example, the first electronic device 102 may be configured to receive a user input indicative of information associated with the health goals of the first user 118. By way of example, and not limitation, the health goals may include, maintenance of one or more of a sleep cycle, a physical fitness, a mental health, social health, or a healthy diet.

In an embodiment, the set of health templates may include one or more health templates that may cater to holistic health goals of a respective user. Each health template may be indicative of the set of activities and the set of health recommendations for the respective user. The set of activities may include, but are not limited to, a water intake activity, a food intake activity, a sleep activity, a step count, a meditation activity, a yoga activity, a physical exercise, a breathing exercise, a stretching exercise, a sedentary task, a walk, a run, a jog, a cycling activity, a swimming activity, a work-out activity, or a listening to music activity. The set of health recommendations may include, but are not limited to, a recommendation for a first activity for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the user. The set of health templates may include for example, 3, 5, 10, or any other number of health templates. In an example, the set of health templates for a certain user may include a list of top health templates (for example, top 3 health templates) that may be recommended for the particular user, based on the user profile information of the particular user.

With reference to FIG. 4A, as shown in the table 400, for example, the user profile information of the “User 1” may indicate that the “User 1” may be a male individual of 30 years age who may reside in Karnataka, India. Further, the user profile information of the “User 1” may indicate that the “User 1” may be a software engineer by profession with an athletic build, a vegan diet, and no medical history. With reference to FIG. 4B, as shown in the table 400, for example, the user profile information of the “User 2” may indicate that the “User 2” may be a female individual of 55 years age who may reside in Delhi, India. Further, the user profile information of the “User 2” may indicate that the “User 2” may be housewife with an overweight body type, a non-vegetarian diet, and a medical history of high blood pressure. As shown in the table 400, for example, three health templates each for physical fitness of respective users (such as, “User 1” and “User 2”) are provided. For example, a first set of health templates including a first health template “T-1”, a second health template “T-2”, and a third health template “T-3” may be provided for the “User 1”. Further, a second set of health templates including and a fourth health template “T-4”, a fifth health template “T-5”, and a sixth health template “T-6” may be provided for the “User 2”.

With reference to FIG. 4A, for example, the first health template “T-1” for the “User 1” may include a 2200 kcal diet based on dietician inputs and a workout including 30 minutes exercise at 7 AM and 15 minutes jogging at 5.30 PM. The first health template “T-1” may further indicate breaks such as, a 20 second eye break every hour and a 2 minute stretch every 2 hours between 10 AM to 5 PM, a water intake of 0.4 to 0.5 liters every 2 hours between 8 AM to 8 PM, and a sleep of 7 hours. Further, the second health template “T-2” for the “User 1” may indicate a 2400 kcal diet based on dietician inputs and a workout including 40 minutes exercise at 7 AM and 20 minutes jogging at 5.30 PM. The second health template “T-2” may indicate breaks such as, a 20 second eye break every hour and a 2 minute stretch every 2 hours between 10 AM to 5 PM, a water intake of 0.5 to 0.6 liters every 2 hours between 8 AM to 8 PM, and a sleep of 7 hours. Further, the third health template “T-3” for the “User 3” may indicate a 2000 kcal diet based on dietician inputs and a workout including 25 minutes exercise at 7 AM and 10 minutes jogging at 5.30 PM. The third health template “T-3” may further indicate breaks such as, a 20 second eye break every hour and a 2 minute stretch every 2 hours between 10 AM to 5 PM, a water intake of 0.3 to 0.4 liters every 2 hours between 8 AM to 8 PM, and a sleep of 7 hours.

With reference to FIG. 4B, for example, the fourth health template “T-4” for the “User 2” may indicate a 2000 kcal diet based on dietician inputs and a workout including 30 minutes meditation at 7 AM and 30 minutes brisk walk at 5.30 PM. The fourth health template “T-4” may further indicate breaks such as, watching TV for maximum of 2 hours and a 5 minute walk every 2 hours between 10 AM to 5 PM, a water intake of 0.4 to 0.5 liters every 2 hours between 8 AM to 8 PM, and a sleep of 8 hours. Further, the fifth health template “T-5” for the “User 2” may indicate a 2100 kcal diet based on dietician inputs and a workout including 35 minutes meditation at 7 AM and 35 minutes brisk walk at 5.30 PM. The fifth health template “T-5” may further indicate breaks such as, watching TV for maximum of 2 hours and a 7 minute walk every 2 hours between 10 AM to 5 PM, a water intake of 0.5 to 0.6 liters every 2 hours between 8 AM to 8 PM, and a sleep of 8 hours. Also, the sixth health template “T-6” for the “User 2” may indicate a 1900 kcal diet based on dietician inputs and a workout including 25 minutes meditation at 7 AM and 20 minutes brisk walk at 5.30 PM. The sixth health template “T-6” may further indicate breaks such as, watching TV for maximum of 2 hours and a 3 minute walk every 2 hours between 10 AM to 5 PM, a water intake of 0.3 to 0.4 liters every 2 hours between 8 AM to 8 PM, and a sleep of 8 hours.

The three number health templates in the set of health templates for each user, as shown in the table 400 of FIGS. 4A and 4B is presented merely as an example. Each set of health templates may include more or less than three health templates, without departure from the scope of the disclosure. It should be noted that data provided in the table 400 may merely be taken as experimental data for exemplary purpose and may not be construed as limiting the present disclosure.

FIGS. 5A and 5B are diagrams that collectively illustrate exemplary operations for control of a personalized health assistant, in accordance with an embodiment of the disclosure. FIGS. 5A and 5B are explained in conjunction with elements from FIGS. 1, 2, 3, 4A, and 4B. With reference to FIGS. 5A and 5B, there is shown a timeline 500 that illustrates exemplary operations from 502 to 534. The exemplary operations may be executed by any computing system, for example, by the first electronic device 102 and/or the server 104 of FIG. 1 or by the circuitry 202 and/or the circuitry 302 of FIG. 2.

At 502, user profile information may be acquired at the first electronic device 102. In an embodiment, the circuitry 202 of the first electronic device 102 may be configured to receive the user profile information associated with the first user 118 of the first electronic device 102 and thereby acquire the user profile information. The user profile information may be received or acquired from a data source other than the one or more sensors 114. The data source may include, for example, the memory 204 associated with the first electronic device 102, a cloud server, an API (i.e., Application Programming Interface), a data aggregator, and the like. In an embodiment, the first electronic device 102 may be configured to receive user inputs indicative of the user profile information associated with the first user 118. The received user profile information may include, but not limited to a gender, an age, a height, a body weight, a body mass index (BMI), a location, food habits, medical data, or health goals of the first user, as described, for example, in FIGS. 4A and 4B.

At 504, the user profile information associated with the first user 118 may be transmitted by the first electronic device 102. In an embodiment, the circuitry 202 of the first electronic device 102 may be configured to transmit the user profile information associated with the first user 118 to the server 104. In an embodiment, the server 104 may be configured to receive the user profile information associated with the first user 118 from the first electronic device 102.

At 506, a set of health templates may be determined at the server 104. In an embodiment, the server 104 may be configured to determine the set of health templates from a stored plurality of health templates, based on an application of the second AI model 110 on the received user profile information associated with the first user 118. The plurality of health templates may be stored in the memory 304 associated with the server 104. The received user profile information associated with the first user 118 may be fed as an input to the second AI model 110. The second AI model 110 may analyze the received user profile information associated with the first user 118, and based on the analysis, the second AI model 110 may determine the set of health templates from the plurality of stored health templates as an output for the input user profile information. In some embodiments, based on the application of the second AI model 110, the circuitry 302 of the server 104 may retrieve the set of health templates from the plurality of health templates stored in the memory 304 of the server 104. Thereafter, the server 104 may transmit the determined set of health templates (as top ‘N’ health templates) to the first electronic device 102 associated with the first user 118. In an embodiment, each of the determined set of health templates may indicate a set of activities and a set of health recommendations for the first user 118 of the first electronic device 102. The determined set of health templates may indicate best or appropriate activities and/or health-related recommendations based on the user profile information as shown, for example, in FIGS. 4A-4B. In some embodiments, the plurality of health templates may be stored in the memory 204 of the first electronic device 102, and the circuitry 202 of the first electronic device 102 may determine the set of health templates from the stored plurality of health templates based on the received user profile information.

At 508, the set of health templates may be received transmitted by the server 104 to the first electronic device 102, based on the user profile information associated with the first user 118. In an embodiment, the circuitry 202 of the first electronic device 102 may be configured to receive the set of health templates from the server 104, based on the user profile information (i.e. associated with the first user 118) that may be transmitted to the server 104.

At 510, a first health template may be selected from the received set of health templates at the first electronic device 102. In an embodiment, the circuitry 202 of the first electronic device 102 may be configured to select the first health template from the received set of health templates. The first electronic device 102 may be configured to receive (via the I/O device 206) a first user input 510A from the first user 118. The first user input 510A may be indicative of a selection of the first health template from the received set of health templates. Based on the received first user input 510A, the first electronic device 102 may be configured to select the first health template from the received set of health templates. Additionally, or alternatively, the first electronic device 102 may be configured to select the first health template from the received set of health templates based on a set of pre-defined rules 510B associated with the first electronic device 102. Examples of the set of pre-defined rules 510B may include one or more health template selection criteria that may be based on at least one of, but not limited to, a gender, an age, a height, a body weight, a body mass index (BMI), a location, food habits, medical data, or health goals of the first user 118.

In an embodiment, the server 104 may be configured to determine the first health template, based on an application of the second AI model 110 on the user profile information associated with the first user 118 received from the first electronic device 102. The second AI model 110 may fed the user profile information associated with the first user 118 as an input for the determination of the first health template. The second AI model 110 may analyze the user profile information based on the set of pre-defined rules 510B associated with the first electronic device 102. Based on the analysis, the second AI model 110 may select the first health template from the set of health templates. Thereafter, the server 104 may transmit the selected first health template to the first electronic device 102 associated with the first user 118.

At 512, the first AI model 108 may be re-trained at the first electronic device 102. In an embodiment, the circuitry 202 of the first electronic device 102 may be configured to re-train the first AI model 108. The first electronic device 102 may be configured to receive a second user input from the first user 118. Herein, the second user input may be indicative of one or more feedbacks 512B associated with the received set of health templates or with the selected first health template. For example, from the first user 118, the first electronic device 102 may receive the one or more feedbacks 512B to the set of health templates or to the selected first health template (based on one or more health tracking operations, which may be performed by the first electronic device 102). By way of example, and not limitations, the one or more feedbacks 512B may include a modification of at least one activity or health recommendation in the received set of health templates. For example, the selected health template or any other health template from the received set of health templates may indicate recommended activities such as, a 2200 kcal diet, 30 minutes exercise at 7 AM, 15 minutes jogging at 6 PM, 10 minutes meditation at 10 PM, a water intake of 3 liters per day, and a sleep of at least 7 hours. Based on a preference of the first user 118, the first user 118 may provide the second user input corresponding to the one or more feedbacks 512B. For instance, the second user input may indicate feedbacks for the selected health template, for example, to modify certain activities such as, a 2300 kcal diet, 20 minutes exercise at 6.40 AM, 20 minutes jogging at 6.30 PM, 5 minutes meditation at 10.30 PM, a water intake of 2.5 liters per day, and a sleep of at least 6 hours.

In certain embodiments, the first electronic device 102 may be configured to determine the one or more feedbacks 512B based on a determination of a set of activities and a set of health parameters associated with the first user 118, and the selected first health template. The determination of the set of activities and the set of health parameters associated with the first user 118, is described, for example, at 522 in FIG. 5A. The first electronic device 102 may determine a deviation of the determined set of activities or the determined set of health parameters from a certain health goal in the selected first health template. The one or more feedbacks 512B may be determined based on the determined deviation of the determined set of activities or the determined set of health parameters from the particular health goals in the selected first health template. An example of a planned calorie consumption (based on the selected first health template) versus an actual calorie consumption of the first user 118 is provided in Table 1, as follows:

TABLE 1
Planned versus actual calorie consumption of the first user 118

| | Breakfast | Lunch | Dinner |
|---|---|---|---|
| Time | 8:30AM | 1:00PM | 8:00PM |
| Planned (as per Health Template) | 400 kcal | 900 kcal | 800 kcal |
| Actuals (monitored) | 460 kcal | 1200 kcal | 940 kcal |

In another example, planned exercise and water intake activities (based on the selected first health template) versus actual exercise and water intake activities of the first user 118 are provided in Table 2, as follows:

Planned versus actual exercise and water intake of the first user 118

| | Exercise | | Water intake | |
|---|---|---|---|---|
| Time | 7:00AM | 5:00PM | 10:30AM | 12:30PM |
| Planned (as per | 30 minutes | 30 minutes | 500 ml | 500 ml |
| Health Template) | | | | |
| Actuals (monitored) | 20 minutes | 20 minutes | 300 ml | 450 ml |

In yet another example, a planned eye break activity (based on the selected first health template) versus an actual eye break activity of the first user 118 is provided in Table 3, as follows:

TABLE 3
Planned versus actual eye break activity of the first user 118

| | Eye break | | |
|---|---|---|---|
| Time | 9:30AM | 10:30AM | 11:30AM |
| Planned (as per Health Template) | 20 seconds | 20 seconds | 20 seconds |
| Actuals (monitored) | 30 seconds | 0 second | 45 seconds |

In yet another example, a planned stretch break activity (based on the selected first health template) versus an actual stretch break activity of the first user 118 is provided in Table 4, as follows:

TABLE 4
Planned versus actual stretch break activity of the first user 118

| | Stretch break | |
|---|---|---|
| Time | 10:30AM | 12:30PM |
| Planned (as per Health Template) | 2 minutes | 2 minutes |
| Actuals (monitored) | 1.5 minutes | 3 minutes |

An example of the one or more feedbacks 512B determined as a deviation of the monitored activities of the first user 118 from the recommended (i.e., planned) activities for the first user 118 (as per the selected first health template) is presented in Table 5, as follows:

TABLE 5
Exemplary values of the one or more feedbacks 512B associated with the first user
118 based on the deviation of the monitored activities from the planned J activities

| | Exercise | Food Intake | Eye Break | Stretch Break | Water |
|---|---|---|---|---|---|
| | (minutes) | (kcal) | (minutes) | (minutes) | Intake (ml) |
| Total-Day | 60 | 2100 | 6 | 14 | 3000 |
| (planned as per | | | | | |
| health template) | | | | | |
| Total-Day | 40 | 2600 | 4 | 16 | 2800 |
| (monitored | | | | | |
| actuals) | | | | | |
| Deviation-Day | −20 | 500 | −2 | 2 | −200 |
| (one or more | | | | | |
| feedbacks 512B) | | | | | |

With reference to the table 5, the one or more feedbacks 512B may indicate that the time for exercise for the first user 118 may require reduction by 20 minutes from 60 minutes to 40 minutes and the calorie consumption may require increasing by 500 kcal from 2100 kcal to 2600 kcal. Further, the one or more feedbacks 512B may indicate that the eye break may require reduction by 2 minutes, the stretch break may require increasing by 2 minutes, and the water intake may require decreasing by 200 ml. It should be noted that data provided in the Tables 1, 2, 3, 4, and 5 may merely be taken as experimental data for exemplary purpose and may not be construed as limiting the present disclosure.

In an embodiment, the first electronic device 102 may augment a first training dataset associated with the first AI model 108 based on at least one of the selection of the first health template (for example, a selection of the first health template 512A determined based on the first user input 510A), the received set of health templates, and the user profile information associated with the first user 118. The first electronic device 102 may augment a second training dataset associated with the first AI model 108 based on at least one of the selection of the first health template 512A, the one or more feedbacks 512B (as per Table 5), and the user profile information associated with the first user 118.

In a re-training phase, the first electronic device 102 may re-train the first AI model 108 by use of training data including different combinations of the augmented first training dataset and the augmented second training dataset. The re-training of the first AI model 108 may include an update of a set of parameters of the first AI model 108 based on whether an output of the final layer for a given input (from the training data) matches a correct result based on a loss function for the first AI model 108. For example, the set of parameters may include weights, regularization parameters, and the like. The above process may be repeated for the same or a different input from the training data till a minima of loss function may be achieved and a training error may be minimized. Several methods for training are known in art, for example, gradient descent, stochastic gradient descent, batch gradient descent, gradient boost, meta-heuristics, and the like. For example, based on the re-training of the first AI model 108, one or more activities or health-related recommendations in the selected first health template 512A may be updated based on the one or more feedbacks 512B (i.e. deviations indicated in Tables 1-5).

At 514, the second AI model 110 may be re-trained at the server 104. In an embodiment, the server 104 may be configured to re-train the second AI model 110. In an embodiment, from the first electronic device 102, the server 104 may receive the selection of the first health template 512A. Further, the first electronic device 102 may be configured to transmit the second user input corresponding to the one or more feedbacks 512B to the server 104 which is trained on the second AI model 110 configured to determine the set of health templates (i.e. described at 506). In other words, from the first electronic device 102, the server 104 may receive information indicative of the one or more feedbacks 512B associated with the determined set of health templates or associated with the selected first health template. The server 104 may re-train the second AI model 110 at least based on the received information indicative of the one or more feedbacks 512B or based on the selection of the first health template 512A. For example, the server 104 may augment a training dataset (for example a third training dataset) associated with the second AI model 110 based on at least one of the received selection of the first health template 512A, the set of health templates, and the user profile information associated with the first user 118. Further, the server 104 may augment a fourth training dataset associated with the second AI model 110 based on at least one of the received one or more feedbacks 512B, the selected first health template, and the user profile information associated with the first user 118. In a re-training phase, the server 104 may re-train the second AI model 110 by use of training data including different combinations of the third training dataset and the fourth training dataset. The re-training of the second AI model 110 may be similar to the re-training of the first AI model 108, described further, for example, at 512.

For example, the second AI model 110 in the server 104 may generate or store the plurality of health templates (for example 100 health templates) for major demographics of the world. For instance, out of the 100 health templates, 40 health templates may be for a particular region (like a particular country, for example for Indian users). The demographics of the Indian users may be divided into multiple types based on 4 regions or zones in the country. As an example, the 4 regions or zones may include a Northern zone, an Eastern zone, a Western zone, and a Southern zone. The 40 health templates for the Indian users may be divided zone-wise, for example, 10 health templates per zone. Each such health template may indicate recommended food intake, diet plan, activities plan (as per FIGS. 4A-4B) according to dietician/physician inputs for users of the corresponding demographics and zone. The server 104 may transmit the generated/retrieved set of health templates to electronic devices of users based on the demographics of the users. Once the health templates may be received by an electronic device of a certain user, the electronic device may receive user input indicative of a selection of a certain health template from the received set of health templates (as described, for example, at 510). After the selection of the certain health template, feedbacks corresponding to the selected health templates may be received from the user (as described, for example, at 512). For example, feedbacks may include information such as, an absence of one or more first food items of interest from the heath template or a presence of one or more second food items, that are not preferred by the user, in the particular health template. The first electronic device 102 of the first user 118 may transmit such feedbacks to the server 104. The server 104 may further receive such feedbacks (i.e. one or more feedbacks 512B) from different electronic devices of multiple users and provide the received feedbacks to the second AI model 110. Based on such received feedbacks and the demographics of the users, the second AI model 110 may be re-trained. The second AI model 110 may generate more health templates (i.e. to enhance a number of templates in the plurality of health templates) based on the re-training and the received feedbacks. As the number of users and feedbacks increases, more health templates may be generated, and the existing health templates may be customized for various demographics of users by the server 104 using the second AI model 110. For example, using the received feedbacks, the server 104 may divide (using the re-trained second AI model 110) the plurality of health templates into more number of categories or zones for appropriate determination of the set of health templates from the plurality of health templates based on the user profile information (indicating demographics) of the users. The real-time generation and customization of the plurality of health templates based on the usage of the personalized health templates by large number users and based on their corresponding feedbacks, may indicate a real-time course correction performed by the server 104 (or by the first electronic device 102) in the plurality of health templates.

At 516, auto reminders may be setup at the first electronic device 102. In an embodiment, the circuitry 202 of the first electronic device 102 may be configured to setup a set of periodic auto-reminders, associated with a set of activities for the first user 118, based on the selected first health template. The set of periodic auto-reminders may be setup for the set of activities (i.e. that may be recommended to the first user 118 based on the selected first health template), to achieve health goals and maintain a healthy lifestyle for the first user 118. Conventionally, a significant manual effort may be required by a user to set periodic reminders associated with activities and health goals of the user. For example, manual selection of activities (such as, a walk activity, a water intake, etc.), time periods (for example, certain number of minutes, hours or days) for the reminders, and manual inputs of health goals (such as, a step count or a sleep duration) may be required. The set-up of periodic reminders based on such manual user inputs may require significant time and effort for users, which may not be appreciated by the users. This may also cause user churn, wherein the users may stop use of conventional personal health assistant system/application on corresponding device and may look for other alternative solutions. In contrast, the disclosed first electronic device 102 may be configured to dynamically setup the set of periodic auto-reminders for the recommended set of activities with minimal user inputs. Such periodic auto-reminders may be associated with the set of activities for the first user 118 and may be set based on the selected first health template. For example, the periodic auto-reminders may be associated with the set of activities including, but not limited to, a water intake activity, a food intake activity, a sleep activity, a step count, a meditation activity, a yoga activity, a physical exercise, a breathing exercise, a stretching exercise, a sedentary task, a walk, a run, a jog, a cycling activity, a swimming activity, a work-out activity, or a listening to music activity.

In an example, the first electronic device 102 may be configured to setup a first set of periodic auto-reminders that may be common to the set of health templates. For example, the first set of periodic auto-reminders may include, a water intake activity, a food intake activity, a sleep activity, a breathing exercise, a stretching exercise, breaks, a walk, or jogging. In an example, a water intake reminder may be setup for every 2 hours, an eye break reminder may be set for every hour, a stretching exercise reminder may be set for every 2 hours, and/or a food intake reminder may be set up for every 4 hours for the first user 118.

At 518, auto reminders may be generated by the server 104. In an embodiment, the circuitry 302 of the server 104 may be configured to generate information about the set of periodic auto-reminders associated with the set of activities for the first user 118. The server 104 may receive the selected first health template from the first electronic device 102. The server 104 may then generate information about the set of periodic auto-reminders associated with the set of activities for the first user 118, based on an application of the second AI model 110 on the selected first health template.

At 520, the generated information about the set of periodic auto-reminders may be transmitted by the server 104. In an embodiment, the circuitry 302 of the server 104 may be configured transmit the generated information about the set of periodic auto-reminders to the first electronic device 102. The first electronic device 102 may be further configured to receive the generated information about the set of periodic auto-reminders from the server 104.

At 522, a set of health parameters and set of activities may be determined at the first electronic device 102. In an embodiment, the circuitry 202 of the first electronic device 102 may be configured to determine, by the one or more sensors 114 associated with the first electronic device 102, at least one of a set of health parameters of the first user 118 or a set of activities of the first user 118, based on the selected first health template. Examples of the one or more sensors 114 may include, but are not limited to, an accelerometer, an altimeter, a gyroscope, a step-count tracker, a heart rate tracker, a pulse rate monitor, a blood oxygen concentration monitor, a bioimpedance sensor, a location sensor, an activity tracker, an ultra-violet (UV) sensor, an electrodermal activity sensor, a skin temperature sensor, an electrocardiography sensor, a gesture sensor, or a magnetometer. The accelerometer may be employed to track movements (such as a forward, a backward, or a side-ways movement) of the first user 118, sense gravity, and determine orientation, or position associated with the first user 118. The altimeter may be employed to determine change in altitude of the first user 118. This may allow to detect whether the first user 118 is climbing or going down a slope or stairs, thereby determine calorie count. The gyroscope may be employed to measure an angular velocity of the first user 118 in order to track and detect motion of the first user 118 accurately. The heart rate tracker may detect heart beats per minute of the first user 118. Such tracker may use light to detect a speed of blood flow on a wrist of the first user 118. The blood oxygen concentration monitor may be employed to measure blood oxygen levels of the first user 118. The bioimpedance sensor may be employed to measure a respiratory rate, a sleep duration, a sleep depth, or a water intake level of the first user 118. The electrocardiography sensor may be employed to detect minute electrical impulse transmitted by the heart of the first user 118. The gesture sensor may be employed to detect gestures of the first user 118 and instruct the first electronic device 102 to perform certain operations, such as, detect activities or provide health recommendations associated with the first user 118. The skin temperature sensor may be employed to detect a body temperature of the first user 118. The detailed implementation of the aforementioned one or more sensors may be known to one skilled in the art, and therefore, a detailed description for the aforementioned one or more sensors has been omitted from the disclosure for the sake of brevity.

For example, the set of health parameters of the first user 118 may include, but are not limited to, a body temperature, a heart rate, a pulse rate, a blood oxygen level, a blood pressure, a blood sugar level, a stress level, a sleep duration, or a depth of sleep. Examples of the set of activities of the first user 118 may include, but are not limited to, a water intake, a food intake, a sleep, a step count, a meditation activity, a yoga activity, a physical exercise, a breathing exercise, a stretching exercise, a sedentary task, a walk, a run, a jog, a cycling activity, a swimming activity, a work-out activity, or a listening to music activity. In an example, the first electronic device 102 may be configured to determine, by the one or more sensors 114 (such as, the accelerometer, gyroscope, or location sensor), movements associated with the first user 118 such as, a walk, a run, a jog, a cycling activity, a swimming activity, a work-out activity, or a listening to music activity. In another example, the first electronic device 102 may be configured to determine, by the one or more sensors 114 (such as skin temperature sensor), whether the first user 118 may be suffering from fever (i.e. health parameter). In another example, the first electronic device 102 may determine, by the one or more sensors 114 (such as heart rate tracker or pulse rate monitor), whether the first user 118 faces issues related to heart or breathing (i.e. health parameter). Such real-time tracking of the health parameters and the activities by the one or more sensors 114 associated with the disclosed first electronic device 102 may help to monitor health progress of the first user 118 with respect to the suggested activities and/or health-related recommendations (such as physical, mental, diet-related plans) indicated in the first health template selected by the first user 118.

In an embodiment, the first electronic device 102 may be configured to apply the first AI model 108 on at least one of the determined set of health parameters or the determined set of activities. Based on the application of the first AI model 108, the first electronic device 102 may be configured to determine a health condition of the first user 118. For example, in case the determined set of health parameters indicates a high body temperature (such as, 99 degree Fahrenheit), the first electronic device may be configured to determine the health condition of the first user 118 as fever.

At 524, information about the determined set of health parameters and the determined set of activities associated with the first user 118 may be transmitted by the first electronic device 102. In an embodiment, the circuitry 202 of the first electronic device 102 may be configured to transmit the information about the set of health parameters and set of activities associated with the first user 118 to the server 104. In an embodiment, the server 104 may receive, from the first electronic device 102, the information about at least one of the set of health parameters of the first user 118 or the set of activities of the first user 118, determined or monitored based on the first health template selected from the set of health templates.

In certain embodiments, the server 104 may be configured to determine the one or more feedbacks 512B based on the received information about the determined set of activities and/or the set of health parameters associated with the first user 118, and based on the selected first health template. The determination of the set of activities and the set of health parameters associated with the first user 118, is described, for example, at 522 in FIG. 5A. The server 104 may determine the deviations of the determined set of activities or the determined set of health parameters from a certain health goal in the selected first health template. The one or more feedbacks 512B may be determined based on the determined deviations of the determined set of activities or the determined set of health parameters from the particular health goals indicated in the selected first health template. Examples of the one or more feedbacks 512B are provided, for example, at 512 in FIG. 5A. The second AI model 110 in the server 104 may be re-trained based on the one or more feedbacks 512B, as described, for example, at 514 in FIG. 5A.

At 526, a set of health recommendations may be determined at the first electronic device 102. In an embodiment, the circuitry 202 of the first electronic device 102 may be configured to determine the set of health recommendations associated with the first user 118. The set of health recommendations may be determined based on an application of the first AI model 108 on the selected first health template and on at least one of the determined set of health parameters of the first user 118 or the determined set of activities of the first user 118. The determined set of health parameters or the determined set of activities of the first user 118 may indicate a user behavior towards the selected first health template. The determined set of health recommendations (i.e. as a smart suggestions) may include, but are not limited to, a recommendation for a first activity (such as any physical or mental activity) for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the first user 118. The first electronic device 102 may feed the selected first health template and at least one of the determined set of health parameters of the first user 118 or the determined set of activities of the first user 118 as input to the first AI model 108. Based on the fed input, the first AI model 108 may determine the set of health recommendations as an output.

In an example, the body temperature of the first user 118 may be higher than a normal body temperature. The first electronic device 102 may determine whether the body temperature is higher due to an ambient temperature. In case, the body temperature is determined to be high due to the ambient temperature, the first electronic device 102 may recommend the first user 118 to intake ample amount of water to keep the body hydrated (as health recommendation). The first electronic device 102 may continue to monitor the body temperature (as a health parameter) of the first user 118 for a certain period of time, by use of the one or more sensors 114. Based on the monitoring, if it is determined that the body temperature does not come down, for example, after 24 to 48 hours or any specific period of time, the first electronic device 102 may recommend the first user 118 to consult a physician, such as, a doctor (as health recommendation).

In another example, the first user 118 may frequently complain about a neck pain, fatigue, and the like (i.e. health parameter). The first electronic device 102 may recommend a stretching exercise (i.e. health recommendation) for 2 minutes after every 2 hours to avoid such pain and fatigue. In yet another example, the first user 118 may be stressed out as he/she may be in a seated posture for a long time, such as, 2 hours. The stress may lead to a variation in blood pressure or blood oxygen levels (i.e. health parameter detected by the one or more sensors 114) of the first user 118. In such case, the first electronic device 102 may recommend the first user 118 to drink ample amount of water, take a break, do a stretching exercise, listen to music, or do meditation (i.e. health recommendations). In an example, the first electronic device 102 may also recommend media content (like song, video, movie, URL, etc) based on the determined set of health parameters and/or the determined set of activities.

At 528, the set of health recommendations may be determined at the server 104. In an embodiment, the circuitry 202 server 104 may determine the set of health recommendations associated with the first user 118, based on an application of the second AI model 110 on the selected first health template and on the information about at least one of the received set of health parameters of the first user 118 or the determined set of activities of the first user 118 (i.e. received from the first electronic device 102 at 524 in FIG. 5B). The server 104 may feed the received selected first health template and the information about at least one of the received set of health parameters of the first user 118 or the determined set of activities of the first user 118 to the second AI model 110 as an input. The second AI model 110 may analyze the received input and determine the set of health recommendations as an output.

At 530, the set of health recommendations may be further transmitted by the server 104 for the first electronic device 102. In an embodiment, the circuitry 302 of the server 104 may be configured to transmit information about the set of health recommendations (i.e. which may be determined at the server 104) to the first electronic device 102.

In an embodiment, the first electronic device 102 may be further configured to update the selected first health template based on the determined set of health recommendations (i.e. either determined by the first electronic device 102 or received from the server 104). For example, the determined set of health recommendations may include a consultation with a physician. In such case, the first electronic device 102 may be further configured to update the selected first health template based on the determined set of health recommendations by a setup of an auto-reminder in the selected first health template to schedule a periodic appointment with the physician. The first electronic device 102 may control the display device 116 to display the updated selected first health template for the first user 118. Based on the newly set-up auto-reminder, the first user 118 may schedule an appointment to consult with the physician.

At 532, a notification may be generated at the first electronic device 102. In an embodiment, the circuitry 202 of the first electronic device 102 may be configured to generate a first notification associated with a first activity of the set of activities for the first user 118 based on the set periodic auto-reminders. The first activity may include, but is not limited to, a water intake, a food intake, a sleep, a step count, a meditation activity, a yoga activity, a physical exercise, a breathing exercise, a stretching exercise, a sedentary task, a walk, a run, a jog, a cycling activity, a swimming activity, a work-out activity, or a listening to music activity. In an embodiment, the first electronic device 102 may be configured to generate a second notification indicating the determined health condition of the first user 118. The health condition may include, but is not limited to, a body temperature, a heart rate, a pulse rate, a blood oxygen level, a blood pressure, a blood sugar level, a stress level, a sleep duration, or a depth of sleep.

The first electronic device 102 may further control the display device 116 to display the generated first notification, and the generated second notification or other notification related to other activities, reminders, and/or monitored health conditions of the first user 118. For example, the first electronic device 102 may set a periodic reminder (e.g., an hourly reminder) of a step count and a water intake associated with the first user 118. Accordingly, the first electronic device 102 may display a notification message every hour on the display device 116, where the notification message may include the step count of the first user 118 in the past hour and also remind the first user 118 about the water intake. Also, along with the display of the notification message, the first electronic device 102 may determine a pulse of the first user 118 and recommend media content (for example soothing music, comedy movie, a weblink to a content source) to the first user 118 based on the determined pulse of the first user 118.

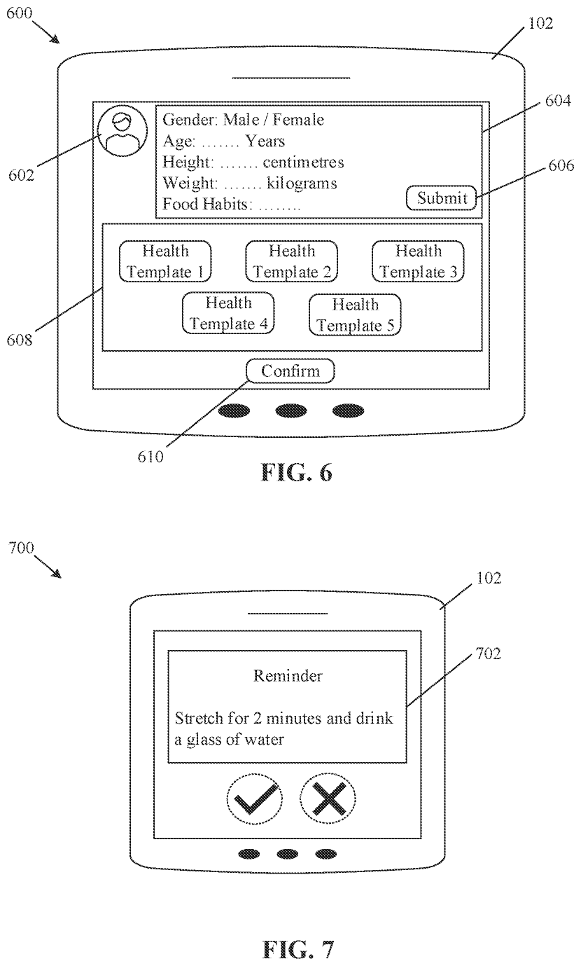

In another example, the first electronic device 102 may determine the health condition of the first user 118 as “fever” based on the determination of the body temperature of the first user 118 by use of the one or more sensors 114. The first electronic device 102 may further recommend one or more activities to the first user 118 as a notification based on the determined health condition, i.e., “fever”. Further, the first electronic device 102 may enable the first user 118 to provide a user input indicative of a completion status of each of the one or more activities. In an embodiment, the first electronic device 102 may receive a user input from the first user 118 indicative of a completion of an activity from the set of activities. Further, the first electronic device 102 may validate the user input indicative of the completion of the activity based on monitoring of the activity by use of the one or more sensors 114 (say in last certain hours, days, or weeks). Accordingly, based on the selected first health template and an application of the first AI model 108, the first electronic device 102 may display a notification of a recommendation for the first user 118, such as, to listen to a light music, consume ample water, or meditate (e.g., based on a recommendation of online media content). The first electronic device 102 may enable the first user 118 to provide the user input indicative of a confirmation of whether the first user 118 has followed the recommendations. By use of the one or more sensors 114, the first electronic device 102 may continue to determine (in real-time) the set of health parameters and the set of activities of the first user 118 to determine if the health condition of the first user 118 has changed or improved. Based on the determined set of health parameters and the set of activities, the first electronic device 102 may validate the confirmation provided by the first user 118. In case the confirmation provided by the first user 118 is invalid, the first electronic device 102 may control the display device 116 to again display the notification of the recommendation along with the determined set of health parameters and the set of activities, for the first user 118. An exemplary user interface associated with a display of the notifications on the first electronic device 102 is described further, for example, in FIG. 7.

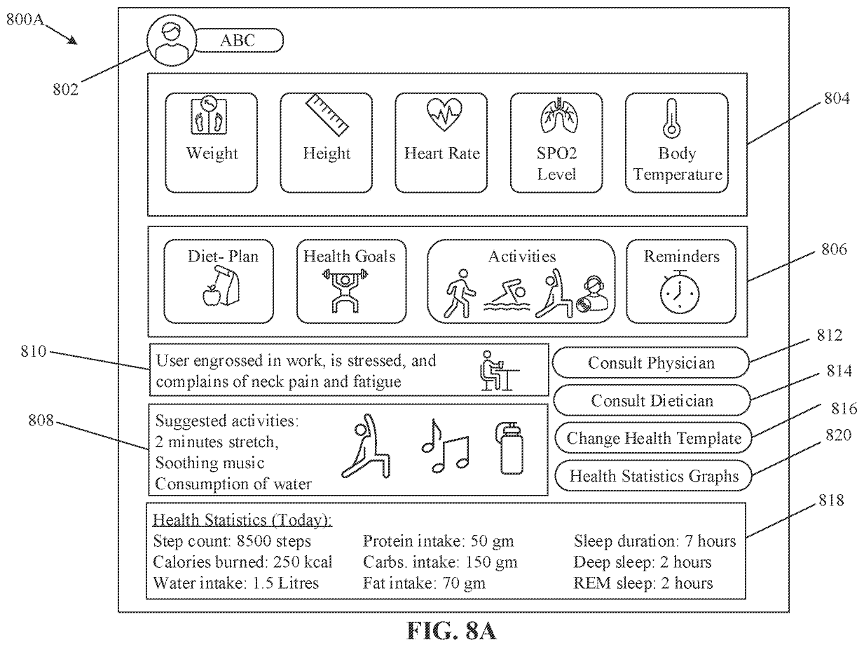

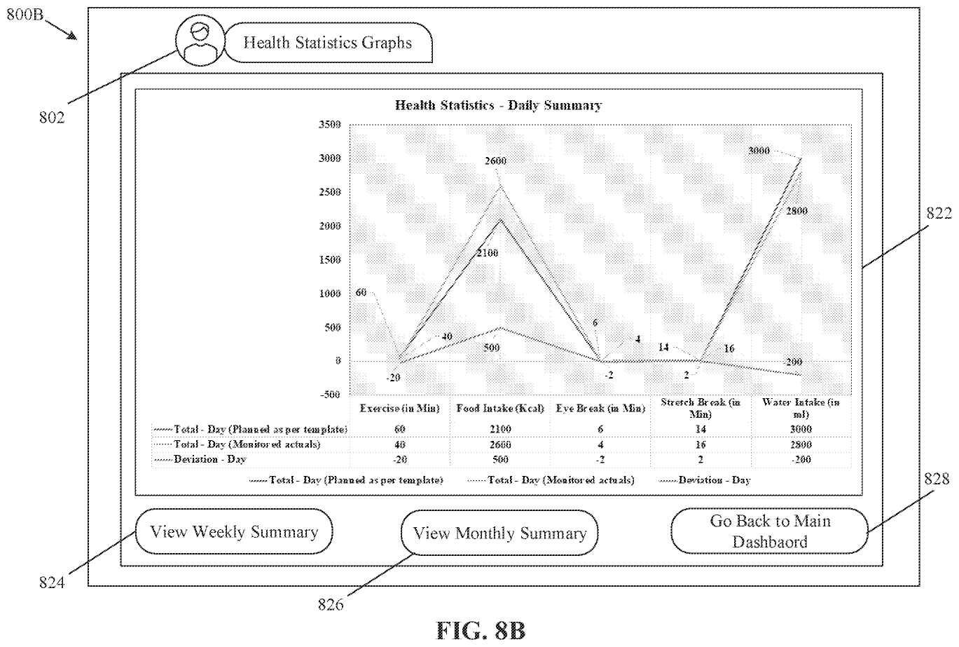

At 534, health information may be displayed. In an embodiment, the circuitry 202 of the first electronic device 102 may be configured to control the display device 116 associated with the first electronic device 102 to render the health information. The health information may be indicative of the determined set of health recommendations and at least one of the determined set of health parameters or the determined set of activities associated with the first user 118. The health information may further indicate statistical information associated with a health of the first user 118. The statistical information associated with the health may include, but is not limited to, an amount of activities performed, an amount of expended calories, information about an endurance, an amount of food intake, an amount of calorie intake, nutrients of consumed food, ingredients of consumed food, an amount of water intake, or a weight change. For example, in FIG. 5B, there is shown health information 534A that may be rendered on the display device 116 associated with the first electronic device 102. The health information 534A may include, but is not limited to, health parameters, activities, health recommendations, statistical information, auto-reminders, completion status of activities, and health conditions associated with the first user 118. An exemplary user interface associated with a display of the health information on the first electronic device 102 is described further, for example, in FIGS. 8A and 8B.

A person skilled in the art would understand that operations 518 and 520 may execute in addition to, or instead of, operation 516, without departure from the scope of the disclosure. Further, operations 528 and 539 may execute in addition to, or instead of, operation 526, without departure from the scope of the disclosure.

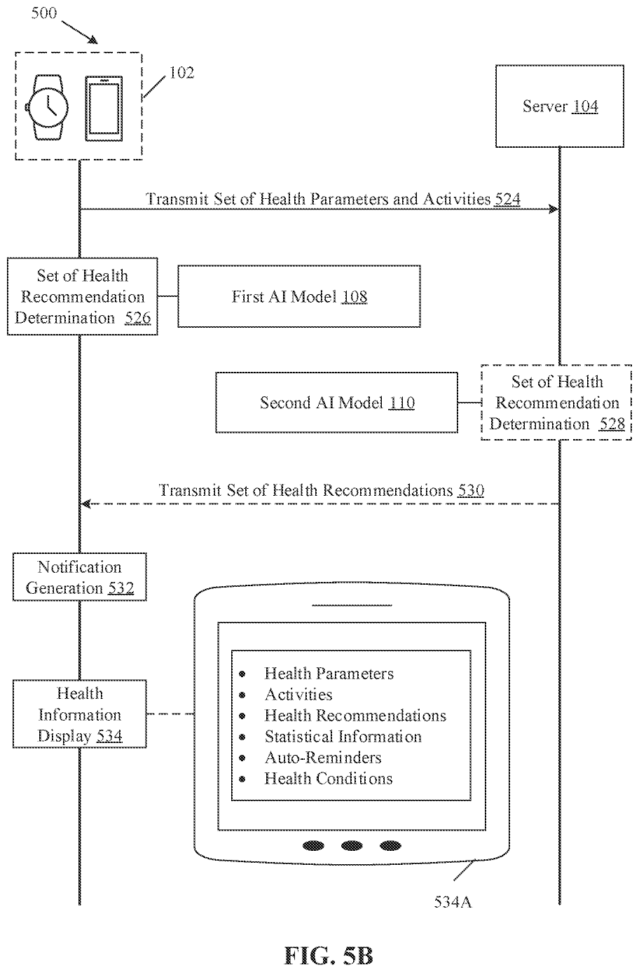

FIG. 6 is a diagram that illustrates an exemplary scenario for control of a personalized health assistant, in accordance with an embodiment of the disclosure. FIG. 6 is explained in conjunction with elements from FIGS. 1, 2, 3, 4A, 4B, 5A, and 5B. With reference to FIG. 6, there is shown an exemplary scenario 600. The scenario 600 may include the first electronic device 102 that may display a plurality of user element (UI) elements through the display device 116. The plurality of UI elements may be associated with a generation of user profile information of a user (such as, the first user 118) and a selection of a first health template from a set of health templates received from the server 104. The plurality of UI elements may include a first UI element 602, a second UI element 604, a third UI element 606, a fourth UI element 608, and a fifth UI element 610. The first electronic device 102 may control the display device 116 to display a user interface (UI). The UI may be an application interface, such as, a user interface of a smartphone application. Alternatively, the UI may be a client interface of a web browser software installed on the first electronic device 102, such as a smartphone.

The first UI element 602 may include a profile picture associated with the first user 118. The second UI element 604 may enable the first electronic device 102 to receive user inputs indicative of the user profile information associated with the first user 118. For example, as shown in FIG. 6, the second UI element 604 may include a set of textboxes to receive user inputs for fields, such as, but not limited to, a gender, an age, a height, a weight, and food habits of the first user 118. Based on the user inputs received through the set of textboxes in the second UI element 604, the first electronic device 102 may generate the user profile information associated with the first user 118. There is further shown, the third UI element 606, such as, a “Submit” button. Based on a user input received though the third UI element 606, the user profile information of the first user 118 may be confirmed. Based on such confirmation, the first electronic device 102 may be configured to transmit the generated user profile information to the server 104.

Based on the transmission of the user profile information to the server 104, the first electronic device 102 may receive the set of health templates from the server 104 or from the memory 204 of the first electronic device 102. The received set of health templates may be based on the user profile information (as described in FIGS. 4A-4B and 5A-5B). The fourth UI element 608 may include a plurality of UI elements, each indicative of a health template of the set of health templates. For example, the plurality of UI elements may be a plurality of buttons to receive a user input of a selection of the first health template from the set of health templates. In FIG. 6, there is shown, the set of health templates including “health template 1”, “health template 2”, “health template 3”, “health template 4” and “health template 5”. For instance, on a certain button of the fourth UI element 608, the first electronic device 102 may receive, from the first user 118, the user input indicative of the selection of the corresponding health template from the set of health templates as the first health template. There is further shown, the fifth UI element 610, such as, a “Confirm” button. In an example, the first electronic device 102 may be configured to receive, from the first user 118, a user input through the “Confirm” button (i.e., the fifth UI element 610) for user confirmation of the selection of the first health template. Based on the user confirmation of the selection of the first health template, the first electronic device 102 may be configured to select the first health template of the set of health templates. It should be noted that the scenario 600 of FIG. 6 is for exemplary purpose and should not be construed to limit the scope of the disclosure. There may be different ways to select the first health template of the set of health templates, without deviation from the scope of the disclosure.

FIG. 7 is a diagram that illustrates an exemplary scenario for display of a completion status of an activity, in accordance with an embodiment of the disclosure. FIG. 7 is explained in conjunction with elements from FIGS. 1, 2, 3, 4A, 4B, 5A, 5B, and 6. With reference to FIG. 7, there is shown an exemplary scenario 700. The scenario 700 may include the first electronic device 102 that may display a set of user element (UI) elements through the display device 116. The set of UI elements may correspond to a completion status of an activity. The set of UI elements may include a first UI element 702 indicative of the first notification associated with a first activity of the set of activities for the first user 118 based on the set periodic auto-reminders. The first notification may include a message such as, but not limited to, “Stretch for 2 minutes and drink a glass of water”.

In an embodiment, the first electronic device 102 may be configured to determine a completion status of the first activity of the first user 118 based on at least one of the determined set of activities of the first user 118 (i.e. determined from the one or more sensors 114 at 522 in FIG. 5A) or a user input received from the first user 118. The first electronic device 102 may be configured to control the display device 116 to display the determined completion status of the first activity. For example, in FIG. 7 there is shown an image representation for the completion status of the first activity, such as a tick mark and a cross mark. In case, the first user 118 completes the first activity, the first electronic device 102 may receive a user input corresponding to the tick mark from the first user 118. On the contrary, if the first user 118 does not complete the first activity, the first electronic device 102 may receive a user input corresponding to the cross mark from the first user 118. For example, after the first user 118 stretches and drinks water as per suggested first activity, the first user 118 may mark the activity as complete through the first UI element 702.

In an embodiment, by use of the one or more sensors 114, the first electronic device 102 may determine the set of health parameters and the set of activities of the first user 118, as described, for example, in FIG. 5A (at 522). Based on the determination of the set of health parameters and the set of activities, the first electronic device 102 may monitor the completion status of the first activity or other activities of the set of activities. In an embodiment, based on the monitored completion status of the first activity, the first electronic device 102 may validate the user input indicative of the completion status of the first activity or other suggested activities as per the first health template. For example, the first electronic device 102 may monitor the stretching activity of the first user 118 by use of sensors such as, but not limited to, an accelerometer, a gyroscope, a heart-rate sensor, a step-count tracker, a pulse rate monitor, and an activity tracker. The first electronic device 102 may monitor the water intake of the first user 118 by use of sensors, such as, but not limited to, a bioimpedance sensor, and a blood oxygen concentration monitor. Based on the validation of the user inputs indicative of the non-completion status of the first activity, the first electronic device 102 may display the first notification again and prompt the first user 118 to complete the first activity. It should be noted that the scenario 700 of FIG. 7 is for exemplary purpose and should not be construed to limit the scope of the disclosure.

FIG. 8A is a diagram that illustrates an exemplary scenario for display of health information, in accordance with an embodiment of the disclosure. FIG. 8A is explained in conjunction with elements from FIGS. 1, 2, 3, 4A, 4B, 5A, 5B, 6, and 7. With reference to FIG. 8A, there is shown an exemplary scenario 800A. The scenario 800A may include the first electronic device 102 that may display a first plurality of user element (UI) elements indicative of the health information, through the display device 116. The first plurality of UI elements may include a first UI element 802, a second UI element 804, a third UI element 806, a fourth UI element 808, a fifth UI element 810, a sixth UI element 812, a seventh UI element 814, an eighth UI element 816, a ninth UI element 818, and a tenth UI element 820.

For example, the first UI element 802 may include a profile picture and a name (such as, “ABC”) associated with the first user 118. Further, the second UI element 804 may indicate first information included in the user profile information associated with the first user 118, and indicate the set of health parameters. For example, as shown in FIG. 8A, the first information may include a weight, and height of the first user 118, and the set of health parameters may include, but not limited to, a heart rate, a SPO₂ level (i.e., a blood oxygen concentration level), and a body temperature of the first user 118.

In an example, the third UI element 806 may indicate second information included in the user profile information associated with the first user 118, the set of activities, and the set of auto-reminders. For example, as shown in FIG. 8A, the second information may include health goals of the first user 118, and a diet plan of the first user 118. There is further shown, the fourth UI element 808 that may be indicative of the first notification associated with one or more activities (like first activity) of the set of activities for the first user 118 based on the set periodic auto-reminders. For example, the first notification may include text such as, “Suggested activities: 2 minutes stretch, soothing music, and consumption of water.” Further, the fifth UI element 810 may be indicative of the second notification indicating the determined health condition of the first user 118. For example, the second notification may include text such as, “User engrossed in work, is stressed, and complains of neck pain and fatigue.” There is further shown, the sixth UI element 812, the seventh UI element 814, and the eighth UI element 816 indicative of the set of health recommendations, for example, “Consult physician”, “Consult Dietician” and “Change health template”, respectively.

The health information may further include statistical information associated with the health of the first user 118. For example, the statistical information associated with the health may include, but is not limited to, at least one of an amount of activities performed, an amount of expended calories, information about an endurance, an amount of food intake, an amount of calorie intake, nutrients of consumed food, ingredients of consumed food, an amount of water intake, or a weight change. The statistical information may also include the recommendations, the tracked activities of the first user 118, and/or the tracked health parameters of the first user 118. For example, as shown in FIG. 8A, the ninth UI element 818 may indicate daily health statistics of the first user 118 including information, such as, but not limited to, a step count (e.g., 8500 steps), calories burnt (e.g., 250 kcal), a water intake (e.g., 1.5 Liters), a protein intake (e.g., 50 grams), a carbohydrate intake (e.g., 150 grams), a fat intake (e.g., 70 grams). The information may further include, but is not limited to, a sleep duration (e.g., 7 hours), a deep sleep duration (e.g., 2 hours) and a REM (Rapid Eye Movement) sleep duration (e.g., 2 hours). Though daily health statistics is shown in FIG. 8A, the statistical information may include data across multiple days, weeks, months, or years for data points related to the health of the first user 118, without departure from the scope of the disclosure. For example, the statistical information may be displayed by use of a graphical representation (like a health dashboard) of the data points related to the health of the first user 118 across various time intervals. The tenth UI element 820 may correspond to a button (such as, a “Health Statistics Graphs” button) that may be used to navigate to another user interface (e.g., a user interface of scenario 800B of FIG. 8B) that may represent the statistical information graphically.

The health information displayed through the first plurality of UI elements may correspond to a health dashboard that may provide a holistic overview of the health of the first user 118. The health information provided by the disclosed first electronic device 102 (or the server 104) may serve as a consolidated cross-functional dashboard including insights that may help to track the health of the first user 118 and also to ascertain a progress of the first user 118 with respect to the desired health goals of the first user 118, as the selected health template (i.e. suggesting the physical activities, mental activities, and/or diet plans) may be personalized for the first user 118. Further, the first user 118 using the disclosed first electronic device 102, may be spared from the manual effort associated with search of several software applications and devices for health tracking, as the disclosed first electronic device 102 may provide a holistic health tracking experience to the first user 118. It should be noted that the scenario 800A of FIG. 8A is for exemplary purpose and should not be construed to limit the scope of the disclosure.

FIG. 8B is a diagram that illustrates an exemplary scenario for graphical display of statistical information included in health information, in accordance with an embodiment of the disclosure. FIG. 8B is explained in conjunction with elements from FIGS. 1, 2, 3, 4A, 4B, 5A, 5B, 6, 7, and 8A. With reference to FIG. 8B, there is shown an exemplary scenario 800B. The scenario 800B may include the first electronic device 102 and a second plurality of user interface (UI) elements including the first UI element 802, an eleventh UI element 822, a twelfth UI element 824, a thirteenth UI element 826, and a fourteenth UI element 828.

For example, the eleventh UI element 822 may indicate a graphical representation of a daily summary of health statistics of the first user 118, based on the statistical information included in the health information of the first user 118 for a certain day. In FIG. 8B and with reference to Table 5 of FIG. 5A, there is shown a graphical and tabular representation of planned activities (as per the selected first health template) versus monitored actual activities of the first user 118, and deviation between the planned activities and the monitored actual activities for a certain day. The activities may include an exercise (in minutes), a food intake (in kcal), an eye break (in minutes), a stretch break (in minutes), and a water intake (in ml). The values represented in graphical and tabular form in the eleventh UI element 822 are same as the values represented in Table 5 for ease of explanation and brevity.

The twelfth UI element 824 may be a button (such as, a “View Weekly Summary” button) that may enable a navigation to another user interface that may display graphical representation of the statistical information across one or more weeks. Further, the thirteenth UI element 826 may be a button (such as, a “View Monthly Summary” button) that may enable a navigation to another user interface that may display graphical representation of the statistical information across one or more months. Also, the UI element 828 may be a button (such as, a “Go Back to Main Dashboard” button) that may enable a navigation to a main user interface (e.g., the user interface of the scenario 800A of FIG. 8A) that may display the health information. It should be noted that the scenario 800B of FIG. 8B is for exemplary purpose and should not be construed to limit the scope of the disclosure.

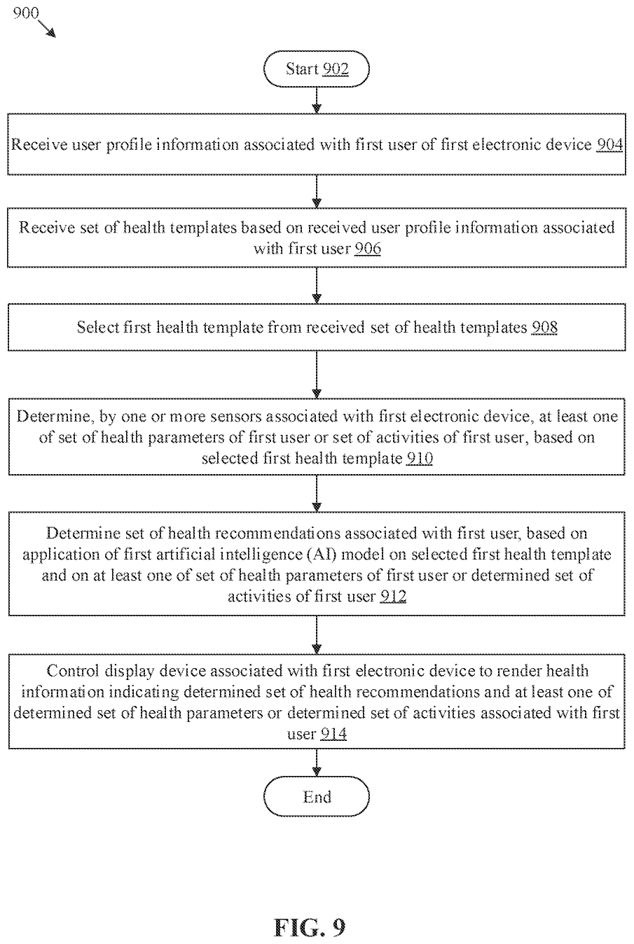

FIG. 9 is a flowchart that illustrates exemplary operations performed by the first electronic device for control of a personalized health assistant, in accordance with an embodiment of the disclosure. FIG. 9 is described in conjunction with elements from FIGS. 1, 2, 3, 4A, 4B, 5A, 5B, 6, 7, 8A, and 8B. With reference to FIG. 9, there is shown a flowchart 900. The flowchart 900 may include operations from 902 to 914 and may be implemented by the first electronic device 102 of FIG. 1 or by the circuitry 202 of FIG. 2. The flowchart 900 may start at 902 and proceed to 904.

At 904, the user profile information associated with the first user of the first electronic device 102 may be received. In an embodiment, the circuitry 202 may be configured to receive the user profile information associated with the first user 118 of the first electronic device 102. The reception of the user profile information is described, for example, in FIG. 5A (at 502).

At 906, the set of health templates may be received based on the received user profile information associated with the first user 118. In an embodiment, the circuitry 202 may be configured to receive the set of health templates based on the received user profile information (associated with the first user 118) from the server 104. The reception of the set of health templates is described, for example, in FIG. 5A (at 506 and 508).

At 908, the first health template from the received set of health templates may be selected. In an embodiment, the circuitry 202 may be configured to select the first health template from the received set of health templates. The selection of the first health template is described, for example, in FIG. 5A at 510).

At 910, at least one of the set of health parameters of the first user 118 or the set of activities of the first user 118 may be determined, based on the selected first health template. In an embodiment, the circuitry 202 may be configured to determine, by the one or more sensors 114 associated with the first electronic device 102, at least one of the set of health parameters of the first user or the set of activities of the first user 118, based on the selected first health template. The determination of the set of health parameters or the set of activities is described, for example, in FIG. 5A (at 522).

At 912, the set of health recommendations associated with the first user 118 may be determined, based on the application of the first artificial intelligence (AI) model 108 on the selected first health template and on at least one of the determined set of health parameters of the first user 118 or the determined set of activities of the first user 118. In an embodiment, the circuitry 202 may be configured to determine the set of health recommendations associated with the first user 118, based on the application of the first artificial intelligence (AI) model 108 on the selected first health template and on at least one of the determined set of health parameters of the first user 118 or the determined set of activities of the first user 118. The determination of the set of health recommendations is described, for example, in FIG. 5B (at 526).

At 914, a display device (e.g., the display device 116) associated with the first electronic device 102 may be controlled, to render the health information indicating the determined set of health recommendations and at least one of the determined set of health parameters or the determined set of activities associated with the first user 118. In an embodiment, the circuitry 202 may be configured to control the display device 116 associated with the first electronic device 102 to render the health information indicating the determined set of health recommendations and at least one of the determined set of health parameters or the determined set of activities associated with the first user 118. The control of the display device 116 is described, for example, in FIG. 5B (at 534) and FIGS. 8A-8B. Control may pass to end.

Although the flowchart 900 is illustrated as discrete operations, such as, 904, 906, 908, 910, 912, and 914 the disclosure is not so limited. Accordingly, in certain embodiments, such discrete operations may be further divided into additional operations, combined into fewer operations, or eliminated, depending on the particular implementation without detracting from the essence of the disclosed embodiments.

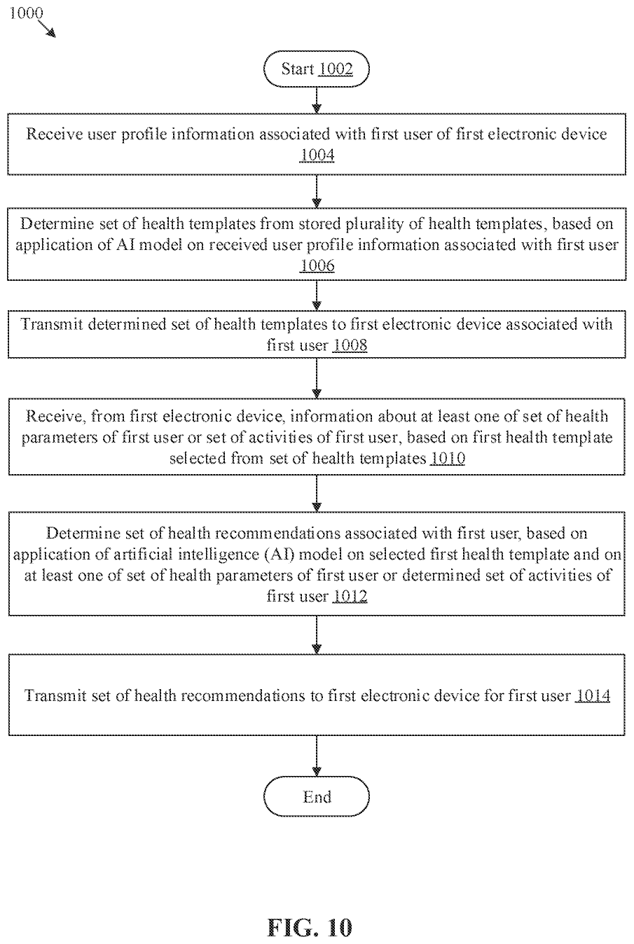

FIG. 10 is a flowchart that illustrates exemplary operations performed by the server for control of a personalized health assistant, in accordance with an embodiment of the disclosure. FIG. 10 is described in conjunction with elements from FIGS. 1, 2, 3, 4A, 4B, 5A, 5B, 6, 7, 8A, 8B, and 9. With reference to FIG. 10, there is shown a flowchart 1000. The flowchart 1000 may include operations from 1002 to 1014 and may be implemented by the server 104 of FIG. 1 or by the circuitry 302 of FIG. 3. The flowchart 1000 may start at 1002 and proceed to 1004.

At 1004, the user profile information associated with the first user 118 of the first electronic device 102 may be received. In an embodiment, the circuitry 302 may be configured to receive the user profile information associated with the first user 118 of the first electronic device 102. The reception of the user profile information is described, for example, in FIG. 5A (at 502 and 504).

At 1006, the set of health templates may be determined from the stored plurality of health templates, based on an application of an AI model (e.g., the second AI model 110) on the received user profile information associated with the first user 118. In an embodiment, the circuitry 302 may be configured to determine the set of health templates from the stored plurality of health templates, based on the application of the second AI model 110 on the received user profile information associated with the first user 118. The determination of the set of health templates is described, for example, in FIG. 5A (at 506).

At 1008, the determined set of health templates may be transmitted to the first electronic device 102 associated with the first user 118. In an embodiment, the circuitry 302 may be configured to transmit the determined set of health templates to the first electronic device 102 associated with the first user 118. The transmission of the set of health templates is described, for example, in FIG. 5A (at 508).

At 1010, information about at least one of the set of health parameters of the first user 118 or the set of activities of the first user 118 may be received, based on the selection of the first health template from the set of health templates. In an embodiment, the circuitry 302 may be configured to receive from the first electronic device 102, information about at least one of the set of health parameters of the first user 118 or the set of activities of the first user 118, based on the first health template selected from the set of health templates. The reception of the information about at least one of the set of health parameters or the set of activities is described, for example, in FIG. 5B (at 522 and 524).

At 1012, the set of health recommendations associated with the first user 118 may be determined, based on an application of an artificial intelligence (AI) model (e.g., the second AI model 110) on the selected first health template and on at least one of the determined set of health parameters of the first user 118 or the determined set of activities of the first user 118. In an embodiment, the circuitry 302 may be configured to determine the set of health recommendations associated with the first user 118, based on application of the second artificial intelligence (AI) model 110 on the selected first health template and at least one of the determined set of health parameters of the first user 118 or the determined set of activities of the first user 118. The determination of the set of health recommendations is described, for example, in FIG. 5B (at 528).

At 1014, the set of health recommendations may be transmitted to the first electronic device 102 for the first user 118. In an embodiment, the circuitry 302 may be configured to transmit the set of health recommendations to the first electronic device 102 for the first user 118. The transmission of the set of health recommendations is described, for example, in FIG. 5B (at 530). Control may pass to end.

Although the flowchart 1000 is illustrated as discrete operations, such as 1004, 1006, 1008, 1010, 1012, and 1014 the disclosure is not so limited. Accordingly, in certain embodiments, such discrete operations may be further divided into additional operations, combined into fewer operations, or eliminated, depending on the particular implementation without detracting from the essence of the disclosed embodiments.

Various embodiments of the disclosure may provide a non-transitory computer-readable medium and/or storage medium having stored thereon, computer-executable instructions executable by a machine and/or a computer to operate a first electronic device (for example, the first electronic device 102). Such instructions may cause the first electronic device 102 to perform operations that include reception of user profile information associated with the first user 118 of the first electronic device 102. The operations may further include reception of a set of health templates based on the received user profile information associated with the first user 118. The operations may further include selection of a first health template from the received set of health templates. The operations may further include determination, by the one or more sensors 114 associated with the first electronic device 102, at least one of a set of health parameters of the first user 118 or a set of activities of the first user, based on the selected first health template. The operations may further include determination of a set of health recommendations associated with the first user 118, based on an application of the first artificial intelligence (AI) model 108 on the selected first health template and on at least one of the determined set of health parameters of the first user 118 or the determined set of activities of the first user 118. The operations may further include control of the display device 116 associated with the first electronic device 102 to render health information indicating the determined set of health recommendations and at least one of the determined set of health parameters or the determined set of activities associated with the first user 118.

Various embodiments of the disclosure may provide a non-transitory computer-readable medium and/or storage medium having stored thereon, computer-executable instructions executable by a machine and/or a computer to operate a server (for example, the server 104). Such instructions may cause the server 104 to perform operations that include storage of a plurality of health templates and a trained artificial intelligence (AI) model (for example the second AI model 110). The operations may further include reception of user profile information associated with a first user (for example the first user 118) of a first electronic device (for example the first electronic device 102). The operations may further include determination of a set of health templates from the stored plurality of health templates, based on an application of the AI model on the received user profile information associated with the first user. The operations may further include transmission of the determined set of health templates to the first electronic device associated with the first user. The operations may further include reception, from the first electronic device, information about at least one of a set of health parameters of the first user or a set of activities of the first user, based on a first health template selected from the set of health templates. The operations may further include determination of a set of health recommendations associated with the first user, based on an application of the AI model on the selected first health template and on the information about at least one of the received set of health parameters of the first user or the determined set of activities of the first user. The operations may further include transmission of the set of health recommendations to the first electronic device for the first user.

Exemplary aspects of the disclosure may provide a first electronic device (such as, the first electronic device 102 of FIG. 1) that includes circuitry (such as, the circuitry 202). The circuitry 202 may be configured to receive user profile information associated with the first user 118 of the first electronic device 102. The circuitry 202 may be further configured to receive a set of health templates based on the received user profile information associated with the first user 118. The circuitry 202 may be further configured to select a first health template from the received set of health templates. The circuitry 202 may be further configured to determine, by the one or more sensors 114 associated with the first electronic device 102, at least one of a set of health parameters of the first user 118 or a set of activities of the first user 118, based on the selected first health template. The circuitry 202 may be configured to determine a set of health recommendations associated with the first user 118, based on application of the first artificial intelligence (AI) model 108 on the selected first health template and at least one of the determined set of health parameters of the first user 118 or the determined set of activities of the first user 118. The circuitry 202 may be configured to control the display device 116 associated with the first electronic device 102 to render health information indicating the determined set of health recommendations and at least one of the determined set of health parameters or the determined set of activities associated with the first user 118.

In an embodiment, the received user profile information comprises at least one of a gender, an age, a height, a body weight, a body mass index (BMI), a location, food habits, medical data, or health goals of the first user 118. In an embodiment, the circuitry 202 may be configured to transmit the received user profile information to the server 104. The circuitry 202 may be configured to receive the set of health templates from the server 104 based on the transmitted user profile information associated with the first user 118. The server 104 may store a plurality of health templates and the second AI model 110 different from the first AI model 108. The server 104 may be configured to retrieve or determine the set of health templates from the stored plurality of health templates, based on an application of the stored second AI model 110 on the received user profile information.

In an embodiment, the circuitry 202 may be configured to receive a first user input indicative of a selection of the first health template from the received set of health templates. The circuitry 202 may be configured to select the first health template from the received set of health templates based on the first user input received from the first user 118. At least one of the first AI model 108 or the second AI model 110 may be re-trained based on the selection of the first health template.

In an embodiment, the circuitry 202 may be configured to receive a second user input from the first user 118. The second user input may be indicative of one or more feedbacks associated with the received set of health templates or with the selected first health template. The circuitry 202 may be configured to transmit the second user input to the server 104, which may be trained on the second AI model 110 configured to determine the set of health templates based on the user profile information. The server 104 may re-train the second AI model 110 based on the received second user input.

In an embodiment, the circuitry 202 may be configured to select first health template from the received set of health templates based on a set of pre-defined rules associated with the first electronic device 102.

In an embodiment, the circuitry 202 may be configured to update the selected first health template based on the determined set of health recommendations.

In an embodiment, the circuitry 202 may be configured to setup of periodic auto-reminders, associated with the set of activities of the first user 118, based on the selected first health template.

In an embodiment, the circuitry 202 may be configured to generate a first notification associated with a first activity of the set of activities of the first user 118 based on the set periodic auto-reminders. The circuitry 202 may be further configured to control the display device 116 to display the generated first notification.

In an embodiment, the circuitry 202 may be configured to determine a completion status of a first activity of the set of activities of the first user 118 based on at least one of the determined set of activities of the first user 118 or a user input received from the first user 118. The circuitry 202 may be configured to control the display device 116 to display the determined completion status of the first activity.

In an embodiment, the circuitry 202 may be configured to apply the first AI model 108 on at least one of the determined set of health parameters or the determined set of activities. The circuitry 202 may be configured to determine a health condition of the first user 118 based on the application of the first AI model 108 on the at least one of the determined set of health parameters or the determined set of activities. The circuitry 202 may be configured to generate a second notification indicating the determined health condition of the first user 118. The circuitry 202 may be configured to control the display device 116 to display the generated second notification.

In an embodiment, the set of activities may include at least one of a water intake, a food intake, a sleep, a step count, a meditation activity, a yoga activity, a physical exercise, a breathing exercise, a stretching exercise, a sedentary task, a walk, a run, a jog, a cycling activity, a swimming activity, a work-out activity, or a listening to music activity.

In an embodiment, the set of health parameters may include at least one of a body temperature, a heart rate, a pulse rate, a blood oxygen level, a blood pressure, a blood sugar level, a stress level, a sleep duration, or a depth of sleep.

In an embodiment, the determined set of health recommendations may include at least one of a recommendation for a first activity for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the first user 118.

In an embodiment, the health information further indicates statistical information associated with a health of the first user 118. The statistical information associated with the health may include at least one of an amount of activities performed, an amount of expended calories, information about an endurance, an amount of food intake, an amount of calorie intake, nutrients of consumed food, ingredients of consumed food, an amount of water intake, or a weight change.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted to carry out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

The present disclosure may also be embedded in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the particular embodiment disclosed, but that the present disclosure will include all embodiments that fall within the scope of the appended claims.

What is claimed is:

1. A first electronic device, comprising:
circuitry configured to:
receive user profile information associated with a first user of the first electronic device;
receive a set of health templates based on the received user profile information associated with the first user;
select a first health template from the received set of health templates;
determine, by one or more sensors associated with the first electronic device, at least one of a set of health parameters of the first user or a set of activities of the first user, based on the selected first health template;
determine a set of health recommendations associated with the first user, based on an application of a first artificial intelligence (AI) model on the selected first health template and on at least one of the determined set of health parameters of the first user or the determined set of activities of the first user; and
control a display device associated with the first electronic device to render health information indicating the determined set of health recommendations and at least one of the determined set of health parameters or the determined set of activities associated with the first user.

2. The first electronic device according to claim 1, wherein the received user profile information comprises at least one of a gender, an age, a height, a body weight, a body mass index (BMI), a location, food habits, medical data, or health goals of the first user.

3. The first electronic device according to claim 1, wherein the circuitry is further configured to:
transmit the received user profile information to a server; and
receive the set of health templates from the server based on the transmitted user profile information associated with the first user,
wherein the server:
stores a plurality of health templates and a second AI model different from the first AI model, and
retrieves the set of health templates from the stored plurality of health templates, based on an application of the stored second AI model on the received user profile information.

4. The first electronic device according to claim 1, wherein the circuitry is further configured to:
receive a first user input indicative of a selection of the first health template from the received set of health templates; and
select the first health template from the received set of health templates based on the first user input received from the first user,
wherein at least one of the first AI model or the second AI model is re-trained based on the selection of the first health template.

5. The first electronic device according to claim 1, wherein the circuitry is further configured to: receive a second user input from the first user, wherein the second user input is indicative of one or more feedbacks associated with the received set of health templates or with the selected first health template; and
transmit the second user input to a server which is trained on a second AI model configured to determine the set of health templates based on the user profile information, wherein the server re-trains the second AI model based on the received second user input.

6. The first electronic device according to claim 1, wherein the circuitry is further configured to select the first health template from the received set of health templates based on a set of pre-defined rules associated with the first electronic device.

7. The first electronic device according to claim 1, wherein the circuitry is further configured to update the selected first health template based on the determined set of health recommendations.

8. The first electronic device according to claim 1, wherein the circuitry is further configured to setup a set of periodic auto-reminders, associated with the set of activities for the first user, based on the selected first health template.

9. The first electronic device according to claim 8, wherein the circuitry is further configured to:
generate a first notification associated with a first activity of the set of activities for the first user based on the set periodic auto-reminders; and
control the display device to display the generated first notification.

10. The first electronic device according to claim 1, wherein the circuitry is further configured to:
determine a completion status of a first activity of the set of activities of the first user based on at least one of the determined set of activities of the first user or a user input received from the first user; and
control the display device to display the determined completion status of the first activity.

11. The first electronic device according to claim 1, wherein the circuitry is further configured to:
apply the first AI model on at least one of the determined set of health parameters or the determined set of activities;
determine a health condition of the first user based on the application of the first AI model on at least one of the determined set of health parameters or the determined set of activities;
generate a second notification indicating the determined health condition of the first user; and
control the display device to display the generated second notification.

12. The first electronic device according to claim 1, wherein the set of activities comprises at least one of a water intake activity, a food intake activity, a sleep activity, a step count, a meditation activity, a yoga activity, a physical exercise, a breathing exercise, a stretching exercise, a sedentary task, a walk, a run, a jog, a cycling activity, a swimming activity, a work-out activity, or a listening to music activity.

13. The first electronic device according to claim 1, wherein the set of health parameters comprises at least one of a body temperature, a heart rate, a pulse rate, a blood oxygen level, a blood pressure, a blood sugar level, a stress level, a sleep duration, or a depth of sleep.

14. The first electronic device according to claim 1, wherein the determined set of health recommendations comprises at least one of a recommendation for a first activity for a first time duration, a recommendation for a first diet plan for a second time duration, a recommendation for a first health goal, a recommendation for an amount of meditation, a recommendation for content, a recommended for a second health template from the received set of health templates, or a recommendation for a consultation with a physician associated with the first user.

15. The first electronic device according to claim 1, wherein the health information further indicate statistical information associated with a health of the first user and wherein the statistical information associated with the health comprises at least one of an amount of activities performed, an amount of expended calories, information about an endurance, an amount of food intake, an amount of calorie intake, nutrients of consumed food, ingredients of consumed food, an amount of water intake, or a weight change.

16. A server, comprising:
a memory configured to store a plurality of health templates and a trained artificial intelligence (AI) model; and
circuitry configured to:
receive user profile information associated with a first user from a first electronic device;
determine a set of health templates from the stored plurality of health templates, based on an application of the AI model on the received user profile information associated with the first user;
transmit the determined set of health templates to the first electronic device associated with the first user;
receive, from the first electronic device, information about at least one of a set of health parameters of the first user or a set of activities of the first user, based on a first health template selected from the set of health templates;
determine a set of health recommendations associated with the first user, based on an application of the AI model on the selected first health template and on the information about at least one of the received set of health parameters of the first user or the determined set of activities of the first user; and
transmit the set of health recommendations to the first electronic device for the first user.

17. The server according to claim 16, wherein the circuitry is further configured to:
generate information about a set of periodic auto-reminders associated with the set of activities for the first user; and
transmit the generated information about the set of periodic auto-reminders to the first electronic device.

18. The server according to claim 16, wherein the circuitry is further configured to:
receive, from the first electronic device, information indicative of one or more feedbacks associated with the determined set of health templates or with the selected first health template; and
re-train the stored AI model based on the received information indicative of the one or more feedbacks.

19. The server according to claim 16, wherein each of the determined set of health templates indicate the set of activities and the set of health recommendations for the first user of the first electronic device.

20. A method, comprising:
in a first electronic device:
receiving user profile information associated with a first user of the first electronic device;
receiving a set of health templates based on the received user profile information associated with the first user;
selecting a first health template from the received set of health templates;
determining, by one or more sensors associated with the first electronic device, at least one of a set of health parameters of the first user or a set of activities of the first user, based on the selected first health template;
determining a set of health recommendations associated with the first user, based on an application of a first artificial intelligence (AI) model on the selected first health template and on at least one of the determined set of health parameters of the first user or the determined set of activities of the first user; and
controlling a display device associated with the first electronic device to render health information indicating the determined set of health recommendations and at least one of the determined set of health parameters or the determined set of activities associated with the first user.
